(19) United States
(12) Patent Application Publication
(10) Pub. No.: US 2011/0044945 A1
(43) Pub. Date: Feb. 24, 2011

(54) AGENTS FOR THE TREATMENT OF MULTIPLE SCLEROSIS AND METHODS OF USING SAME

(75) Inventors: Nathan Karin, Haifa (IL); Gizi Wildbaum, Kiryat Yam (IL); Moran Meiron, Haifa (IL); Yaniv Zohar, Kiryat-Haim (IL)

Correspondence Address: MARTIN D. MOYNIHAN d/b/a PRTSI, INC., P.O. BOX 16446, ARLINGTON, VA, 22215, US

(73) Assignee: Rappaport Family Institute for Research in the Medical Sciences, Haifa (IL)

(21) Appl. No.: 12/449,376

(22) PCT Filed: Feb. 6, 2008
(86) PCT No.: PCT/IL2008/000166

§ 371 Date: Sep. 16, 2010

Related U.S. Application Data
(60) Provisional application No. 60/900,094, filed on Feb. 8, 2007.
(60) Provisional application No. 60/929,591, filed on Jul. 5, 2007.

(51) Int. Cl. A61K 38/21; A61K 35/26; A61K 39/00; A61K 39/385; A61P 25/00; A61J 1/14

(52) U.S. Cl. 424/85.6; 424/93.7; 424/198.1; 424/195.11; 604/404

(57) ABSTRACT

A method of treating Multiple Sclerosis is disclosed. The method comprises administering to the subject a therapeutically effective amount of SDF-1 alpha. An article of manufacture comprising SDF-1 alpha and an anti-Multiple Sclerosis agent is also disclosed.

### FIELD AND BACKGROUND OF THE INVENTION

The present invention relates to the use of SDF-1α for the treatment of multiple sclerosis and compositions thereof.

Chemokines are small (˜8-14 kDa), structurally cytokine-like, secreted proteins that regulate cell trafficking. They are produced and secreted by a wide variety of cell types in response to early inflammatory mediators, such as IL-1β or TNF-α, and in response to bacterial or viral infection. Chemokines function mainly as chemoattractants for leukocytes, recruiting monocytes, neutrophils and other effector cells from the blood to sites of infection or damage. They can be released by many different cell types (e.g. macrophages) and can mediate a range of pro-inflammatory effects on leukocytes, such as triggering of chemotaxis, degranulation, synthesis of lipid mediators, and integrin activation.

Chemokines can be subdivided into four classes, the C—C, C—X—C, C and C-X3-C chemokines, depending on the location of the first two cysteines in their protein sequence. The interaction of these soluble proteins with their specific receptors, which belong to the superfamily of seven-transmembrane domain G-protein-coupled receptors (GPCRs), mediate their biological effects resulting in, among other responses, rapid increase in intracellular calcium concentration, changes in cell shape, increased expression of cellular adhesion molecules, degranulation, and promotion of cell migration.

The Stromal Cell Derived Factor 1 (SDF-1, GenBank Accession Nos. NM_{—}000609 and NM_{—}199168), also referred to as CXCL12, is produced in two forms, SDF-1α/CXCL12a and SDF-1β/CXCL12b, by alternate splicing of the same gene [De La Luz Sierra et al., Blood (2004) 103:2452-2459]. SDF-1α/β is produced by many cell types, including bone marrow stromal cells, astrocytes and endothelial cells, and is constitutively expressed in many tissues including the central nervous system (CNS), thymus, spleen and bone marrow [Bleul et al., J. Exp. Med. (1996) 184:1101-1109]. SDF-1α/β is strongly chemotactic for lymphocytes, including monocytes, bone marrow neutrophils, early-stage B cell precursors and T cells, and is involved in directing the migration of these cells to the different tissues [Pelletier et al., Blood (2000) 96:2682-90]. SDF-1α/β is a co-stimulator of lymphocyte activation [Bleul et al., supra; Nanki and Lipsky, J Immunol (2000) 164:5010-4] and has also been implicated as an important cell coordinator during fetal development [Ma et al., Proc. Nat. Acad. Sci. (1998) 95: 9448-9453].

The main receptor for SDF-1/CXCL12 is CXCR4, also known as fusin or LESTR, although recent reports have suggested that CXCR7 may also bind SDF-1 [Balabanian et al., J Biol Chem (2005) 280:35760-35766]. CXCR4 has a wide cellular distribution, with expression on most immature and mature hematopoietic cell types, including T and B cells, monocytes/macrophages, neutrophils and dendritic cells. In addition, CXCR4 can also be found on vascular endothelial cells and neuronal/nerve cells [Rossi and Zlotnik, Annu Rev Immunol. (2000) 18:217-42].

Various SDF-1α reagents have been designed to date as research tools in order to investigate the role SDF-1α plays in mammalian physiology. Thus, Suzuki et al. disclose a SDF-1α fusion protein composed of murine SDF-1α and the constant region of human IgG. This fusion protein bound specifically to mouse and human CXCR4 and was used in flow cytometry analysis. Their findings have demonstrated the involvement of CXCR4 expression in T cell development in the thymus, particularly in positive selection [Suzuki et al., Int Immunol. (1998) 10(8):1049-56].

In addition, efforts have been made to enhance the pharmacological applications of SDF-1α. For example, U.S. Publication No. 20030171551 discloses chimeric molecules for the stimulation of an anti-tumor immune response. The described chimeric molecules comprise an anti-tumor antibody connected to a chemokine, such as SDF-1, which allows local delivery of chemokines to the tumor site and may aid in the attack against tumors. In the fusion protein described therein, the chemokine is fused to the amino terminus (variable region) of either the heavy or light chain of the antibody.

Multiple sclerosis (MS) is an inflammatory, demyelinating disease of the central nervous system (CNS), characterized by various symptoms of neurological dysfunction. MS and its animal model, experimental autoimmune encephalomyelitis (EAE), are believed to result from autoimmune mediated activated immune cells, such as T- and B-lymphocytes as well as macrophages and microglia, and is considered to be an inflammatory neurodegenerative disease. Pathologically, MS is characterized by perivenous infiltration of lymphocytes and macrophages into the CNS parenchyma, resulting in demyelinative lesions termed plaques. These plaques, which are the hallmark of MS, are associated with oligodendrocytes death, axonal damage and neuronal loss. The etiology of MS has not yet been fully elucidated and it is attributed to both genetic and environmental causes, yet factors which regulate leukocyte entry into the CNS may play a role in MS development as well as in lesion pathogenesis.

There is substantial evidence to support the hypothesis that SDF-1α is involved in MS pathogenesis. For example, it has been reported that in the MS brain, the expression of SDF-1α is up-regulated, particularly by astrocytes but also by monocytes/macrophages [Calderon et al., J Neuroimmunol. (2006) 177(1-2):27-39].

Calderon et al. have indicated that elevation in chemokines within the MS brain is likely to attract dendritic cells, macrophages and T cells to the perivascular areas of the CNS leading to production of inflammatory mediators resulting in oligodendrocyte damage, demyelination, and neuronal injury typical of MS [Calderon et al., supra]. Furthermore, their findings suggest that increased SDF-1α may initiate and augment such inflammatory response. Hence, these findings teach away from using SDF-1α as therapeutics of MS.

U.S. Pat. No. 5,756,084 discloses SDF-1α and SDF-1β DNA and polypeptides which can be used for diagnoses and treatment of diseases including inflammatory diseases, infectious diseases, cancer and neurodegenerative diseases (e.g. multiple sclerosis). However, soluble SDF-1 described in U.S. Pat. No. 5,756,084 has been suggested for both upregulating and downregulating the immune response. No specific guidance is provided for MS. Due to this lack of guidance as well as the general understanding that SDF-1 promotes MS pathogenesis (see Calderon supra), one of ordinary skill in the art would not be motivated to use SDF-1 for treating MS.

U.S. Publication No. 20060257359 discloses means of modulating phenotypes of macrophage related cells for the treatment of diseases, such as multiple sclerosis. Modulating (e.g., increasing or decreasing) the cellular phenotype is accomplished by introducing to macrophage related cells effectors, such as a protein, an antibody or a RNA molecule (e.g., a short interfering RNA), thereby altering gene expression and cell phenotype (e.g., secretion of cytokines or cell migration). SDF-1 is specified therein. However, U.S. Publication No. 20060257359 does not provide any experimental support to indicate treatment of MS.

U.S. Publication No. 20030103938 discloses means of preventing or treating a Th1 or Th2 cell-related disease, by influencing the Th1/Th2 ratio, using IL-2 or IL-4 in combination with SDF-1α. The Th1 cell-related diseases include cancer, infectious diseases and autoimmune diseases (e.g. multiple sclerosis). These inventors showed that the use of IL-4 in combination with SDF-1α can switch non-antigen-specific cord blood CD4⁺ T cells to Th2 cells and thus may influence the cellular response at the site of inflammation (e.g. cytokine production). However, this invention teaches therapeutics by the use of SDF-1 in combination with IL-2 or IL-4 and not as a sole chemokine. Such therapeutics may not be compatible for human treatment as clinical trials have indicated that this strategy promotes the development of allergic diseases [Pedotti et al., Nat Immunol. (2001) 2(3):216-22].

There is thus a widely recognized need and it would be highly advantageous to have SDF-1α fusion proteins that can be used in the treatment of multiple sclerosis.

### SUMMARY OF THE INVENTION

According to one aspect of the present invention there is provided a method of treating Multiple Sclerosis in a subject in need thereof, the method comprising administering to the subject a therapeutically effective amount of SDF-1α, thereby treating Multiple Sclerosis in the subject.

According to another aspect of the present invention there is provided a method of treating Multiple Sclerosis in a subject in need thereof, the method comprising isolating T cells from the subject, subjecting the T cells to treatment with SDF-1α and implanting the SDF-1α treated T cells into the subject, thereby treating Multiple Sclerosis in the subject.

According to further features in preferred embodiments of the invention described below, the subjecting is effected so as to upregulate secretion of IL-10 from the T cells.

According to still further features in the described preferred embodiments the T cells comprise regulatory T cells.

According to still further features in the described preferred embodiments the regulatory T cells comprise CD4⁺CD25⁻FOXp3⁻ T cells.

According to still further features in the described preferred embodiments the subjecting the T cells is further effected in a presence of IL-12 neutralizing antibody.

According to still further features in the described preferred embodiments subjecting the T cells is further effected in a presence of anti-IL-4 neutralizing antibody.

According to yet another aspect of the present invention there is provided a use of SDF-1α for the manufacture of a medicament identified for treating Multiple Sclerosis.

According to yet another aspect of the present invention there is provided an article of manufacture comprising SDF-1α and an anti-Multiple Sclerosis agent being packaged in a packaging material and identified in print, in or on the packaging material for use in the treatment of Multiple Sclerosis.

According to still further features in the described preferred embodiments the SDF-1α is capable of upregulating secretion of IL-10 from macrophages and T cells.

According to still further features in the described preferred embodiments the subject is undergoing an acute attack of Multiple Sclerosis.

According to still further features in the described preferred embodiments an amino acid sequence of the SDF-1α is attached to a heterologous amino acid sequence.

According to still further features in the described preferred embodiments the method does not comprise administering IL-2 or IL-4.

According to still further features in the described preferred embodiments the medicament does not further comprise IL-2 or IL-4.

According to still further features in the described preferred embodiments the anti-Multiple Sclerosis agent is not IL-2 or IL-4.

According to still further features in the described preferred embodiments the method further comprises administering to the subject an additional anti-Multiple Sclerosis agent.

According to still further features in the described preferred embodiments the anti-Multiple Sclerosis agent is selected from the group consisting of Interferon Beta 1a, Interferon Beta 1b, Glatiramer Acetate, Mitoxantrone, MethylPrednisolone, Prednisone, Prednisolone, Dexamethasone, Adreno-corticotrophic Hormone (ACTH) and Corticotropin.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph depicting suppression of EAE by administration of SDF-1α-encoding DNA plasmid. EAE induced mice were subjected to SDF-1α-encoding DNA plasmid (closed circles), a control plasmid encoding β-actin (open squares), an empty vector (closed squares, pcDNA3) or PBS (open circles) as described in Example 1 hereinbelow. An observer blind to the experimental protocol monitored the development and progression of the disease. Results represent 1 of 4 experiments with similar results and are expressed as the mean maximal score ±SE;

FIGS. 2A-D are images of histological sections depicting suppression of EAE by administration of SDF-1α-encoding DNA plasmid. The pictures show histological analysis of lumbar spinal cords of the above described EAE treated mice (of FIG. 1). The table expresses the quantification analysis of these sections. A scale ranging from 0 to 3, based on the number of perivascular lesions per section, was used to quantify the histological score of disease (as described in Example 1 hereinbelow). A representative section from each group is also shown: FIG. 2A represents naïve mice; FIG. 2B represents EAE induced mice; FIG. 2C represents EAE mice subjected to DNA vaccines encoding β-actin; and FIG. 2D represents EAE mice subjected to DNA vaccines encoding SDF-1α. The mean histological score ±SE was calculated for each group.

FIG. 3 is a graph depicting the reversed effect of SDF-1α mAb on suppression of EAE by SDF-1α DNA vaccines. After the onset of EAE, mice were subjected to either PBS (open circles), 50 μg empty plasmid (closed squares), DNA vaccines encoding SDF-1α alone (closed circles, pcSDF-1), or followed by subjection of either anti-SDF-1α mAb (open squares), or control antibody (open triangles) as described in Example 2 hereinbelow. An observer blind to the experimental protocol then monitored the development and progression of the disease. Results are shown as the mean maximal score ±SE.

FIGS. 4A-B are graphs depicting the dual function of SDF-1α in the regulation of EAE. FIG. 4A shows EAE induced mice subjected to PBS″ (open circles), anti-SDF-1α mAb (closed circles), or control antibody (open squares) after the onset of disease (as described in Example 3 hereinbelow). An observer blind to the experimental protocol then monitored the development and progression of the disease. Results are shown as the mean maximal score ±SE. The data shown here represent 1 of 3 experiments with the same results; and FIG. 4B shows EAE induced mice subjected to PBS (open circles), anti-SDF-1α mAb (closed circles.), or control antibody (open squares) beginning 3-4 days before the onset of disease (as described in Example 3 hereinbelow). An observer blind to the experimental protocol then monitored the development and progression of the disease. Results are shown as the mean maximal score ±SE. The depicted results are of 1 of 3 independent consecutive experiments.

FIGS. 5A-I are graphs depicting the functional polarization of macrophages and T cells directed by SDF-1α. FIGS. 5A-C are graphs showing the effect of SDF-1α on cytokine production by primary spleen cells responding to their target antigen (MOGp₃₅₋₅₅); FIGS. 5D-F are graphs showing the effect of SDF-1α on cytokine production by freshly isolated peritoneal macrophages stimulated with LPS; FIGS. 5G-I are graphs showing the effect of SDF-1α on cytokine production by anti-CD3-activated CD4⁺ T cells (purified from the spleens of naïve donors). Secreted levels of IL-10 (FIGS. 5A, D, G), IL-12 (FIGS. 5B, E), TNF-α (FIGS. 5C, F, H), and IL-2 (FIG. 5I) were each measured by ELISA. Results are shown as mean triplicates ±SE.

FIGS. 6A-B are graphs depicting the effect of SDF-1α on monocytes following neutralizing of the CXCR4 by specific monoclonal antibodies. FIG. 6A shows SDF-1α-induced IL-10 production by THP-1 monocytes following subjection of cells to mAb CXCR4; and FIG. 6B shows SDF-1α-induced THP-1 cell migration in a TansWell system following subjection of cells to mAb CXCR4. The depicted results are 1 out of 3 experiments with very similar observations and are shown as mean (triplicates) ±SE.

FIG. 7 is a picture depicting the SDF-1α-Ig fusion protein of this invention. The picture shows western blot analysis of SDF-1α-Ig fusion protein under reducing (with β-mercaptoethanol, +β-me) and non-reducing conditions (without β-mercaptoethanol, −β-me).

FIGS. 8A-C are bar graphs depicting the biological activity preserved by SDF-1α-Ig. FIG. 8A shows migration assay of THP-1 (human monocytic cells). Lower chambers of Transwells were supplemented with (a) culture media, (b) rSDF-1α, (c) SDF-1α-Ig or (d) β-actin-Ig. Results are shown as mean (triplicates) of the migration percentage (number of cells that migrated to the lower chamber divided by the number of cells originally plated in the upper chamber) ±SE; FIG. 8B shows IL-10 secretion by peritoneal macrophages supplemented with (a) PBS, (b) rSDF-1α, (c) SDF-1α-Ig or (d) β-actin-Ig. Results of triplicates were measured by ELISA and are shown as mean triplicates ±SE; and FIG. 8C shows IL-10 secretion by primary splenocytes responding to their target MOGp₃₅₋₅₅ antigen supplemented with (a) PBS, (b) rSDF-1α, (c) SDF-1α-Ig or (d) or β-actin-Ig. Results of triplicates were measured by ELISA and are shown as mean triplicates ±SE.

FIG. 9 is a graph depicting SDF-1α-Ig suppression of ongoing EAE. EAE induced mice were subjected to SDF-1α-Ig (closed circles), β-actin-Ig (open squares) or PBS (open circles) as described in Example 7 hereinbelow. An observer blind to the experimental protocol monitored the development and progression of the disease. Results are expressed as the mean maximal score ±SE;

FIGS. 10A-H are images of histological sections depicting SDF-1α-Ig suppression of ongoing EAE. The pictures show histological analysis of lumbar spinal cords of the above described EAE treated mice (of FIG. 9). A scale ranging from 0 to 3, based on the number of perivascular lesions per section, was used to quantify the histological score of disease (as described in Example 1 hereinbelow). The table presents the quantification analysis of these sections. A representative section from each group of mice is shown: FIG. 10A represents naïve mice; FIG. 10B represents EAE induced mice; FIG. 10C represents β-actin-Ig treated EAE mice; and FIG. 10D represents SDF-1α-Ig treated EAE mice. Sequential sections were also subjected to immunohistochemistry of IL-10 expression: FIG. 10E represents naïve mice; FIG. 10F represents EAE induced mice; FIG. 10G represents β-actin-Ig treated EAE mice; and FIG. 10H represents SDF-1α-Ig treated EAE mice.

FIGS. 11A-G are bar graphs depicting SDF-1α-Ig influence on cytokine secretion by EAE derived splenocytes. EAE splenocytes derived from EAE mice (top bar) β-actin-Ig treated EAE mice (middle bar) or SDF-1α-Ig treated EAE mice (bottom bar). After 24 hours in the presence of MOGp35-55 cytokine secretion levels were measured by ELISA. FIG. 11A shows IL-10 secretion; FIG. 11B shows IL-4 secretion; FIG. 11C shows TGF-β secretion; FIG. 11D shows IL-12 secretion; FIG. 11E shows IL-17 secretion; FIG. 11F shows IL-23 secretion; and FIG. 11G shows TNF-α secretion. Results are shown as the mean of triplicates ±SE.

FIGS. 12A-C are images of IL-10 immunohistochemistry depicting SDF-1α-Ig influence in EAE derived splenocytes. FIG. 12A represents EAE mice; FIG. 12B represents β-actin-Ig-treated EAE mice; and FIG. 12C represents SDF-1α-Ig-treated EAE mice.

FIGS. 13A-F are histograms of FACS analysis depicting SDF-1α-Ig influence on IL-10 expression in EAE derived splenocytes. Intracellular staining of IL-10 was performed on macrophages (CD11b⁺ cells, FIGS. 13B, D and F) and on CD4⁺ T cells (FIGS. A, C and E). FIGS. 13A-B represent EAE control mice; FIGS. 13C-D represent β-actin-Ig treated EAE mice; and FIGS. 13E-F represent SDF-1α-Ig treated EAE mice.

FIG. 14A is a line graph depicting the long term effect of SDF-1α-Ig on full-blown EAE. After the onset of long-term form of EAE (45 days) mice were subjected to SDF-1α-Ig (open circles), β-actin-Ig (open squares) or PBS (close circles) and monitored for the development and progression of disease by an observer blind to the experimental protocol. Results of 1 out of 3 independent experiments is depicted as the mean maximal score ±SE.

FIGS. 14B-C are bar graphs depicting the effect of SDF-1α-Ig on proliferation and IL-2 secretion of T cells. Just before the peak of disease (day 24) primary T cells from the cervical lymph nodes of control mice (a, top bar), β-actin treated mice (b, middle bar) or SDF-1α-Ig treated mice (c, bottom bar) were subjected to MOGp35-55 induced activation. FIG. 14B depicts the proliferative response; FIG. 14C depicts levels of IL-2 secretion.

FIGS. 14D-F are histograms of FACS analysis depicting the effect of SDF-1α-Ig on the expression of Annexin V in PI-CD4+ T cells. FIG. 14D depicts CD4+ T cells from control EAE mice; FIG. 14E depicts CD4+ T cells from EAE mice treated with β-actin-Ig; and FIG. 14F depicts CD4+ T cells from EAE mice treated with SDF-1α-Ig.

FIG. 15 is a graph depicting suppression of EAE by transfer of donor derived antigen-specific T cells (of SDF-1α treated EAE mice). EAE mice were adoptively transferred, at the onset of disease, as follows: recipient group administered T cells isolated from protected SDF-1α-Ig treated EAE mice (closed squares), recipient group administered T cells isolated from β-actin-Ig treated EAE mice (closed circles), and recipients injected with PBS (open squares). All groups were monitored for the development and progression of the disease by an observer blind to the experimental protocol. Shown are results representing one out of three experiments with similar data. Results are shown as the mean maximal score ±SE.

FIGS. 16A-B are histograms of FACS analysis depicting expression of CD25 and FoxP3 in donor derived IL-10^{high} T cells. FIG. 16A depicts CD25 expression in IL-10^{high} T cells; and FIG. 16B depicts FOXp3 expression in IL-10^{high} T cells.

FIG. 17 is a bar graph depicting the ability of donor derived IL-10^{high} T cells to suppress the proliferative response of antigen specific primary T cells. The figure depicts the proliferative response of: primary T cells from control EAE mice (105 per well, lane a); primary T cells from protected mice (105 per well, lane b); primary T cells from protected mice (104 per well, lane c); primary T cells from protected mice (104 per well) in addition to primary T cells from control EAE mice (105 per well, lane d); and primary T cells from protected mice (104 per well) in addition to primary T cells from control EAE mice (105 per well) and anti IL-10 mAb (lane e).

FIGS. 18A-D are line graphs depicting the dependency of SDF-1α-Ig therapy on IL-10. FIGS. 18A, C depict two separate experiments in IL-10^{-/-} mice; FIGS. 18B, D depict two separate experiments in IL-10^{+/+} mice. Mice were treated with PBS (closed circles), β-actin-Ig (closed squares), or SDF-1α-Ig (open squares). Results of both independent experiments with similar data (6 mice per group in each experiment) are shown as mean EAE score ±SE.

FIGS. 19A-E are histograms of FACS analysis depicting the effect of SDF-1α-Ig on redirecting the polarization of antigen specific effector (Th1) cells into IL-10 producing regulatory T cells. MOGp35-55 CD4+ T cell line was selected during two subsequent stimulation cycles in the presence of the target antigen and the combination of recombinant mouse IL-12 and anti-IL-4 neutralizing antibodies. Subsequently these cells were activated in cultures supplemented with SDF-1α-Ig (FIGS. 19C, E) or without SDF-1α-Ig (FIGS. 19A, B and D). Cells were subjected to intracellular staining of cytokines as illustrated.

FIGS. 20A-E are bar graphs depicting the effect of SDF-1α-Ig on redirecting the polarization of antigen specific effector (Th1) cells into IL-10 producing regulatory T cells. MOGp35-55 CD4+ T cell line was selected during two subsequent stimulation cycles in the presence of the target antigen and the combination of recombinant mouse IL-12 and anti-IL-4 neutralizing antibodies. Subsequently these cells were activated in cultures supplemented with SDF-1α-Ig (lane b) or without SDF-1α-Ig (lane a). Secretion of various cytokines was detected by ELISA.

FIG. 21 is a line graph depicting the suppressor effect of SDF-1α-Ig polarized IL-10 producing regulatory T cells on EAE. MOGp35-55 CD4+ T cell line was selected during two subsequent stimulation cycles in the presence of the target antigen and the combination of recombinant mouse IL-12 and anti-IL-4 neutralizing antibodies. Subsequently these cells were detected for their competence to suppress ongoing EAE. Results depict mice treated by PBS (open circles), mice treated by control effector T cells (open squares) and mice treated by SDF-1α-Ig treated cells (closed squares). Results of one out of 2 independent experiments with similar data are shown as mean EAE score ±SE.

### DESCRIPTION OF THE PREFERRED EMBODIMENTS

The present invention is of methods for treating Multiple Sclerosis using SDF-1α.

The principles and operation of the method according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Multiple sclerosis (MS) is an inflammatory, demyelinating disease of the central nervous system (CNS), characterized by various symptoms of neurological dysfunction. MS and its animal model, experimental autoimmune encephalomyelitis (EAE), are believed to result from autoimmune mediated activated immune cells, such as T- and B-lymphocytes as well as macrophages and microglia, and is considered to be an inflammatory neurodegenerative disease. Pathologically, MS is characterized by perivenous infiltration of lymphocytes and macrophages into the CNS parenchyma, resulting in demyelinative lesions termed plaques. These plaques, which are the hallmark of MS, are associated with oligodendrocytes death, axonal damage and neuronal loss. The etiology of MS has not yet been fully elucidated and it is attributed to both genetic and environmental causes, yet factors which regulate leukocyte entry into the CNS may play a role in MS development as well as in lesion pathogenesis.

It has been previously reported that SDF-1α, which is a strong chemoattractant and co-stimulator for lymphocytes, is constitutively expressed, at low levels, in the healthy CNS [Bleul et al., supra]. Additionally, the expression of SDF-1α has been reported to be up-regulated in the MS brain [Calderon et al., J Neuroimmunol. (2006) 177(1-2):27-39].

Whilst reducing the present invention to practice the present inventors have unexpectedly discovered that SDF-1α can be used to suppress active and ongoing MS. These results contradicted previous conceptions of SDF-1 acting as a pro-inflammatory mediator in MS, initiating and enhancing inflammatory responses in the CNS [Calderon et al., supra].

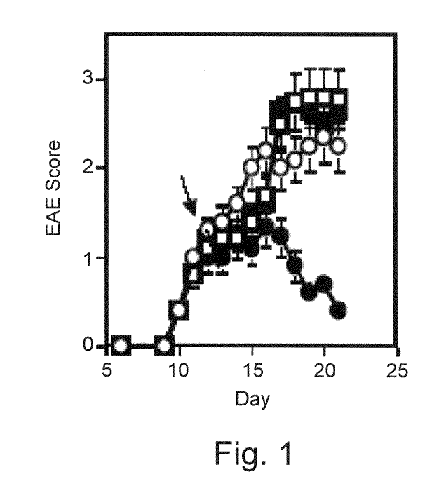

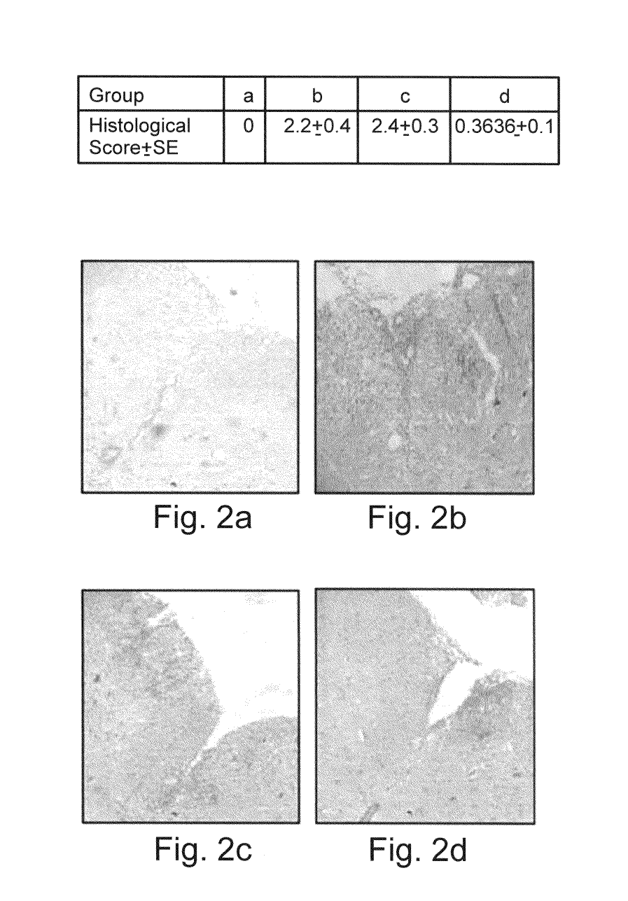

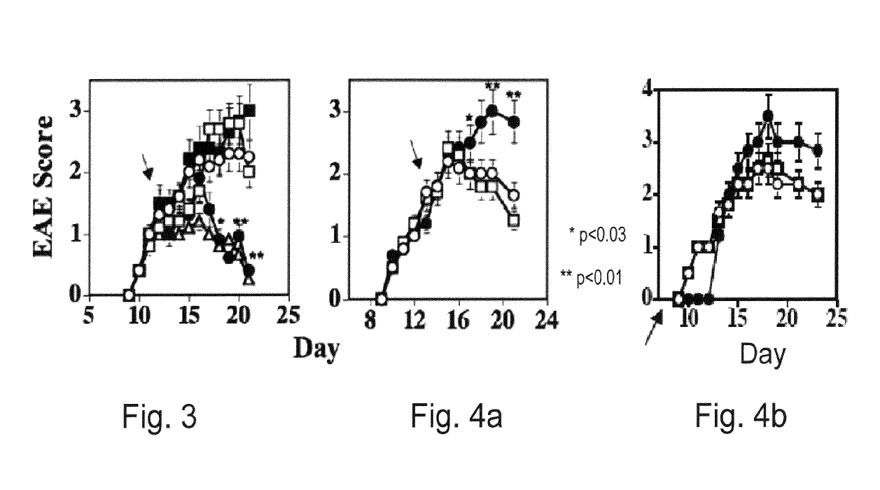

As is illustrated herein below and the Examples section which follows, SDF-1α polypeptides generated according to the teachings of the present invention were shown to be therapeutic for the treatment of MS as was manifested by suppression of ongoing encephalomyelitis (EAE) in vivo. Subjection of EAE induced mice to SDF-1α (i.e., targeted DNA plasmid encoding SDF-1α) resulted in remission of active disease as measured by EAE score and by histological score (see FIGS. 1 and 2A-D). Subjection of SDF-1α treated mice to SDF-1α neutralizing antibodies reversed the therapeutic effect of SDF-1α and resulted in severe and active disease (see FIG. 3). Moreover, neutralizing SDF-1α during ongoing EAE was shown to aggravate disease manifestation (see FIG. 4A). These findings prove that up-regulation of SDF-1α serves as a beneficial goal in the treatment of MS.

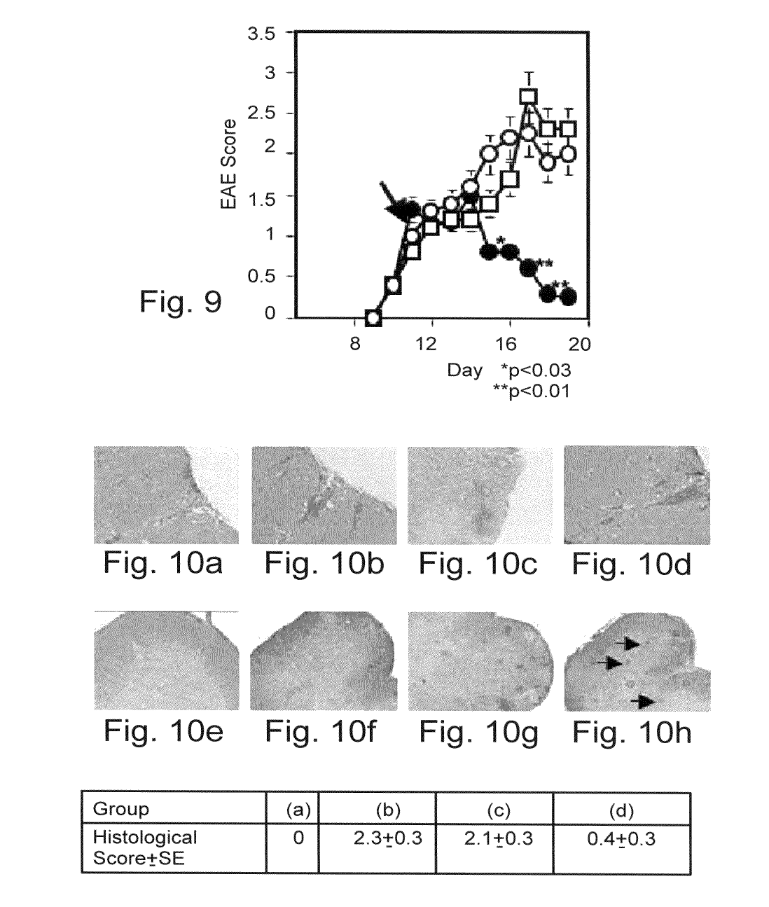

Furthermore, the present inventors have constructed SDF-1α fusion polypeptides and expressed them in mammalian cell systems (see Example 6 of the Examples section which follows). Functionality of the SDF-1α fusion protein was shown by its chemoattractant properties (see FIG. 8A), as well as its ability to elicit IL-10 production in macrophages and T cells (see FIGS. 8B and 8C). Administration of SDF-1α-Ig fusion protein to EAE induced mice resulted in remission of active disease as measured by EAE score (see FIG. 9) and by histological score (see FIG. 10D).

Taken together the present teachings portray a therapeutic value for SDF-1α and suggest the use of same for the treatment of MS.

Thus, according to one aspect of the present invention there is provided a method of treating Multiple Sclerosis in a subject in need thereof, the method comprising administering to the subject a therapeutically effective amount of SDF-1α, thereby treating Multiple Sclerosis in the subject.

As used herein the term “treating” refers to preventing, curing, reversing, attenuating, alleviating, minimizing, suppressing or halting the deleterious effects of Multiple Sclerosis.

As used herein the phrase “Multiple Sclerosis” refers to the inflammatory, demyelinating disease of the central nervous system (CNS) which is typically characterized by various symptoms of neurological dysfunction. Any type of Multiple Sclerosis may be treated according to the teachings of the present invention including relapsing-remitting, secondary progressive, primary progressive, progressive relapsing and special cases of MS with non-standard behavior (also referred to as borderline forms of MS), such as for example without limitation, Neuromyelitis optica (NMO), Balo concentric sclerosis, Schilder disease, Marburg multiple sclerosis, acute disseminated encephalomyelitis (ADEM) and autoimmune variants of peripheral neuropathies. The disease may be treated at any stage although preferably the disease is treated when the subject is undergoing an acute attack.

As used herein the phrase “a subject in need thereof” refers to a mammal, preferably a human subject who has been diagnosed with probable or definite multiple sclerosis, e.g., a subject who experienced one neurological attack affecting the CNS and accompanied by demyelinating lesions on brain magnetic resonance imaging (MRI). The neurological attack can involve acute or sub-acute neurological symptomatology (attack) manifested by various clinical presentations such as without limitation, unilateral loss of vision, vertigo and sensory loss. The diagnosis of probable MS can also include laboratory tests involving evaluation of IgG synthesis and oligoclonal bands in the cerebrospinal fluid (CSF) which provide evidence of chronic inflammation of the central nervous system.

As used herein the term “SDF-1α” (stromal cell-derived factor-1 alpha) refers to at least an active portion of a mammalian (e.g., human) C—X—C chemokine polypeptide (also designated CXCL12) having at least one functional property of SDF-1α (e.g., chemotaxis or binding to CXCR4). Preferably the SDF-1α of the present invention is capable of down-regulation of at least one pro-inflammatory cytokine (e.g., IL-12 and TNF-α) and/or up-regulation of at least one anti-inflammatory cytokine (e.g. IL-10) as further described herein below. Preferably, the SDF-1α of the present invention is capable of suppressing on-going MS as described in FIGS. 1 and 9 (see the Examples section which follows). Examples of SDF-1α amino acid sequences are set forth in SEQ ID NO: 2 or 10 and in GenBank Accession Nos. NP_{—}000600, NP_{—}001029058, NP_{—}954637 (encoded by GenBank Accession Nos. NM_{—}000609 and NM_{—}199168).

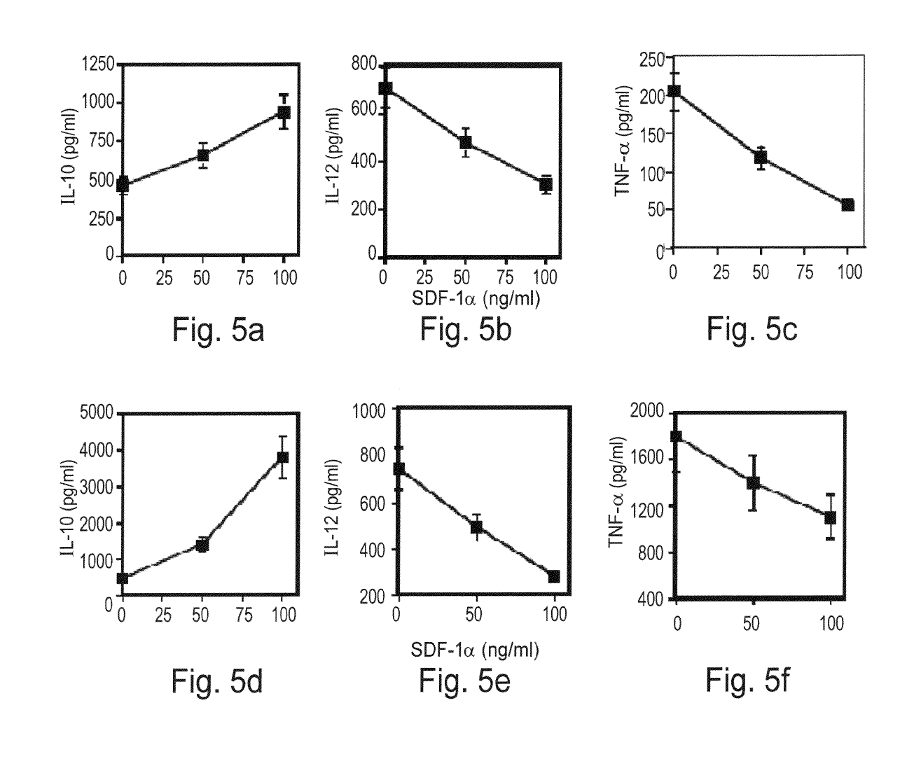

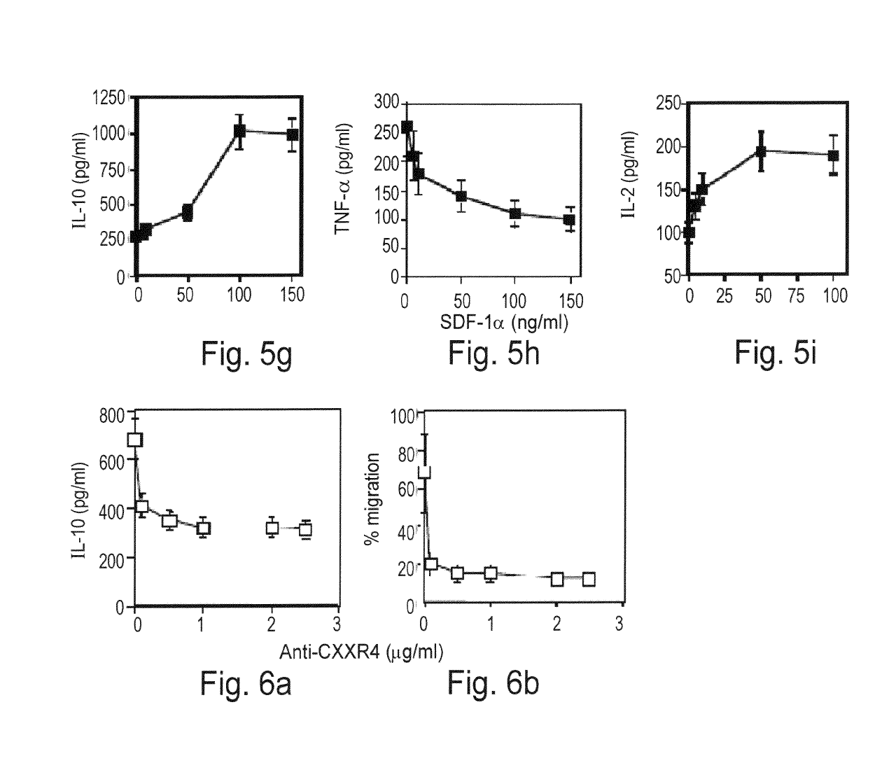

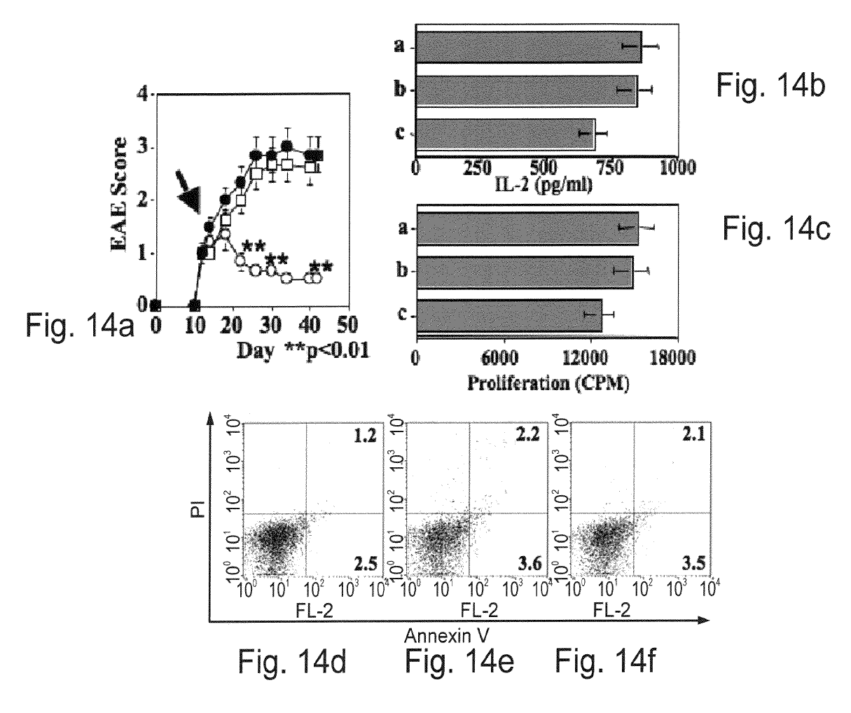

As mentioned, the SDF-1α polypeptide of the present invention is preferably capable of down-regulating at least one pro-inflammatory cytokine (e.g., IL-12 and TNF-α) and/or up-regulating at least one anti-inflammatory cytokine (e.g. IL-10). Without being bound to theory, the present inventors contemplate that the mechanism behind SDF-1α's anti-MS activity may involve at least one of the following: (1) down-regulation of macrophage generated pro-inflammatory cytokine production (e.g., IL-12 and TNF-α; see FIGS. 5E and 5F) and up-regulation of macrophage generated anti-inflammatory cytokine (IL-10) production (see FIG. 5D); (2) up-regulation of T cell generated anti-inflammatory cytokine (IL-10) production (see FIG. 5G); and (3) selection of IL-10-producing regulatory T cells (Tr1) capable of transferring the beneficial effect of therapy to EAE mice (see FIG. 14 of the Examples section which follows), either directly or via its effect on macrophages.

Thus, according to a preferred embodiment of this aspect of the present invention, the SDF-1α of the present invention is capable of upregulating secretion of IL-10 from macrophages and T cells.

As used herein the term “IL-10” (Interleukin-10) refers to the anti-inflammatory cytokine (i.e., capable of inhibiting synthesis of pro-inflammatory cytokines, such as IL-2), an example of which is set forth by GenBank accession number NM_{—}000572.

As used herein the term “macrophages” refers to phagocytic white blood cells that differentiate from monocytes. Examples of such cells include, without limiting to, macrophages, dendritic cells, microglial cells, Kupffer cells, alveolar macrophages, osteoclasts and any cells of related cell types, such as macrophage cell lines (e.g., THP-1 cells).

As used herein the phrase “T cells” refers to the white blood cells known as T lymphocytes which play a central role in cell-mediated immunity. T cells can include any known cells expressing the T cell receptor (TCR), such as without limiting to, T helper cells (Th), T cytotoxic cells (CTL), T memory cells, Regulatory T cells (Treg), Natural Killer T cells (NKT) and γδ T cells.

Any SDF-1α known in the art can be used in accordance with the teachings of the present invention. For example, recombinant human SDF-1α (CXCL12) is available from ProSpec-Tany TechnoGene Ltd, Catalog No. CHM-262; recombinant human SDF-1α from Cell Sciences, Catalog Nos. CRS000A, CRS000B and CRS000C; and recombinant human SDF-1α, 125I Conjugated/Tagged from PerkinElmer, Catalog Nos. NEX346025UC and NEX346005UC.

According to a particularly preferred embodiment of the present invention, SDF-1α is attached to a heterologous amino acid sequence.

As used herein the phrase “heterologous amino acid sequence” refers to an amino acid sequence which does not endogenously form a part of the SDF-1α amino acid sequence. Preferably, the heterologous amino acid sequence does not down-regulate the biological activity (i.e., anti-MS activity) of the SDF-1α polypeptide.

The heterologous amino acid sequence may serve to ensure stability of the SDF-1α of the present invention without compromising its activity. Thus, for example, the sequence may increase the half-life of the SDF-1α chimeric molecule in the serum. Alternatively, the heterologous amino acid sequence may aid in the isolation of a recombinant SDF-1α as further described herein below. Examples of heterologous amino acid sequences that may be used in accordance with the teachings of the present invention include, but are not limited to, immunoglobulin, galactosidase, glucuronidase, glutathione-S-transferase (GST), carboxy terminal peptide (CTP) from chorionic gonadotrophin (CGβ) and chloramphenicol acetyltransferase (CAT) [see for example Suzuki et al., supra; and U.S. Publication No. 20030171551].

The exact site at which fusion (conjugation) between the heterologous amino acid sequence and the SDF-1α amino acid sequence is not critical. Generally the heterologous amino acid sequence is localized at the amino- or carboxyl-terminus (n-ter or c-ter, respectively) of the SDF-1α polypeptide of the present invention. Particular sites are well known in the art and may be selected in order to optimize the biological activity, secretion or binding characteristics of the chimeric molecules of this aspect of the present invention (see Example 6 of the Example section which follows).

The heterologous amino acid sequence may be attached to the SDF-1α amino acid sequence by any of peptide or non-peptide bond. Attachment of the SDF-1α amino acid sequence to the heterologous amino acid sequence may be effected by direct covalent bonding (peptide bond or a substituted peptide bond) or indirect binding such as by the use of a linker having functional groups. Functional groups include, without limitation, a free carboxylic acid (C(═O)OH), a free amino group (NH₂), an ester group (C(═O)OR, where R is alkyl, cycloalkyl or aryl), an acyl halide group (C(═O)A, where A is fluoride, chloride, bromide or iodide), a halide (fluoride, chloride, bromide or iodide), a hydroxyl group (OH), a thiol group (SH), a nitrile group (C≡N), a free C-carbamic group (NR″—C(═O)—OR′, where each of R′ and R″ is independently hydrogen, alkyl, cycloalkyl or aryl).

It will be appreciated that treatment of Multiple Sclerosis according to the present invention may be combined with other treatment methods known in the art (i.e., combination therapy). These include, but are not limited to, Interferon Beta 1a, Interferon Beta 1b, Glatiramer Acetate, Mitoxantrone, MethylPrednisolone, Prednisone, Prednisolone, Dexamethasone, Adreno-corticotrophic Hormone (ACTH) and Corticotropin.

According to one embodiment of this aspect of the present invention, the method of the present invention is not accompanied by administering IL-2 or IL-4.

As used herein the term “IL-2” (Interleukin-2) refers to the pro-inflammatory cytokine (i.e., capable of initiating an immune response such as T cell growth, differentiation and survival), an example of which is set forth by GenBank accession number NM_{—}000586.

As used herein the term “IL-4” (Interleukin-4) refers to the immunoregulatory cytokine (i.e., capable of suppressing pro-inflammatory cytokine production of activated monocytes) an example of which is set forth by GenBank accession number NM_{—}000589.

SDF-1α of the present invention can be administered to the subject per se, or as part of a pharmaceutical composition, which also includes a physiologically acceptable carrier. The purpose of a pharmaceutical composition is to facilitate administration of the active ingredient to an organism.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the preparation accountable for the intended biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media [Mutter et al. (1979)].

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

The discovery that SDF-1α can polarize T cells to secrete anti-inflammatory cytokines (e.g. IL-10) instead of pro-inflammatory cytokines (e.g., IL-12 and TNF-α) suggests the use of SDF-1α in isolated settings so as to avoid undesired side effects. See Example 11 where SDF-1α-Ig redirects the polarization of antigen specific effector (Th1) cells into IL-10 producing regulatory T cells that suppress multiple sclerosis in a murine model.

Thus, according to another aspect of the present invention there is provided a method of treating Multiple Sclerosis in a subject in need thereof, the method comprising isolating T cells from the subject, subjecting the T cells to treatment with SDF-1α and implanting the SDF-1α treated T cells into the subject, thereby treating Multiple Sclerosis in the subject.

As used herein the term “isolating T cells” refers to the process of removing T cells from a multiple sclerosis affected subject.

Thus isolated T cells can be comprised in a crude blood sample or further purified.

Several techniques are known for isolating T cells (see for example, Leavitt et al., Hum. Gene Ther. 5: 1115-1120 (1994)). The expression of surface markers facilitates identification and purification of T cells. Methods of identification and isolation of T cells include FACS, panning with magnetic beads and human T-cell subset columns. Cells isolated according to the teachings of the present invention should stay sterile and preferably stay out of the body for a minimal time period.

Subsequent to cell isolation, the T cells are subjected to culture in the present of SDF-1α. Such culture conditions are explained in detail in Example 11 (in the Example section hereinbelow). For example, the isolated T cells (about 10×10⁶ cells) may be cultured in the presence of SDF-1α-Ig (50 μg/ml) and a stimulatory peptide (e.g. MOGp35-55 peptide, 50 μg/ml), in a humidified 7.5% CO₂ atmosphere at 37° C. for 72 hours.

The isolated T cells may be cultured in the presence of additional agents. For instance, the T cells may be cultured in the presence of IL-12 neutralizing antibody (e.g., R&D Systems Inc., Minneapolis, Minn.) or in the presence of an anti-IL-4 neutralizing antibody (e.g., R&D Systems Inc., Minneapolis, Minn.). Such culturing conditions enable polarization of the isolated T cells into T regulatory cells rather than into T helper cells.

Preferably, such culturing conditions polarize T cells to exhibit upregulation in IL-10 secretion, to become T regulatory cells or to express CD4⁺CD25⁻FOXp3⁻.

SDF-1α treated T cells are then implanted into the subject (e.g., a subject diagnosed with Multiple Sclerosis as described hereinabove).

Those skilled in the art are capable of determining when and how to implant the T cells to thereby treat Multiple Sclerosis. The implantation can be carried out via local injection, by administration into the systemic (e.g., via the blood stream or the peritoneal cavity) or portal circulation system, or by any other practical means (see for example, WO/2001/078752).

The procedure may be repeated as required, such as during relapse.

Thus, the present invention provides compositions and methods of treating MS using in vivo and ex-vivo settings.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Targeted DNA Vaccines Encoding SDF-1α Suppress Ongoing Experimental Autoimmune Encephalomyelitis (EAE) in Mice without Eliciting Autoantibody Production

Materials and Experimental Procedures

Mice

6-week-old female C57BL/6 mice were purchased from Harlan (Jerusalem, Israel) and maintained under specific pathogen-free conditions.

Peptide Antigens

Myelin oligodendrocyte glycoprotein MOG₃₅₋₅₅ (SEQ ID NO: 15) was constructed by the PAN facility of the Beckman Center of Stanford University. After purification by HPLC, the sequence was confirmed by amino acid analysis and the mass was checked by mass spectroscopy. Purification of the peptide used in by this invention was >95%.

Induction of Active EAE in Mice

Active induction of EAE was induced by immunizing C57BL\6 female mice with MOGp₃₅₋₅₅/CFA as previously described by Tompkins et al. [Tompkins et al., J Immunol (2002) 168:4173-83]. Mice were monitored daily for clinical signs by an observer blind to the treatment protocol. EAE was scored as follows: 0—clinically normal; 1—flaccid tail; 2—hind limb paralysis; 3—total hind limb paralysis, accompanied by an apparent front limb paralysis; 4—total hind limb and front limb paralysis; and 5—death.

Production and Administration of DNA Vaccines

cDNA encoding mouse SDF-1α (GenBank Accession Nos. BC006040 or E09670, SEQ ID NO: 9) was generated by RT-PCR of RNA extracted from mouse splenocytes using the primers: sense, 5′ gctagcATGGACGCCAAGGTCGTCGC 3′ (SEQ ID NO: 11) and antisense, 5′ ctcgagCTTGTTTAAGGCTTTGTCC 3′ (SEQ ID NO: 12). cDNA was cloned into a pcDNA plasmid. Large scale production and purification of SDF-1α DNA vaccines were prepared prior to administration to EAE mice.

Just after the onset of active EAE disease (day 10), C57BL\6 female mice were separated into four groups of mice based on the severity of the disease (6 per group). On days 11, 13, 15, and 17, after the induction of disease, these groups were injected intramuscularly (i.m.) with either SDF-1α-encoding DNA plasmid, a control plasmid encoding β-actin, an empty vector (pcDNA3) or PBS. All plasmids were administered at a concentration of 50 μg/mouse.

Histopathology

On day 21 (from induction of EAE), three representative mice from each group (as explicated above) were euthanized and lumbar spinal cords were removed. Lumbar spinal cords (8 sections/sample) were dissected, fixed in 4% paraformaldehyde, dehydrated and embedded in paraffin. 5-μm thick sections were stained with H&E (Sigma, St. Louis, Mo.). Each section was evaluated for tissue damage and mononuclear infiltration using the following scale: 0—no mononuclear cell infiltration; 1—one to five perivascular lesions per section with minimal parenchymal infiltration; 2—five to 10 perivascular lesions per section with parenchymal infiltration; and 3—more than 10 perivascular lesions per section with extensive parenchymal infiltration.

Results

Vaccination of EAE induced mice with plasmid DNA encoding SDF-1α just after the onset of disease (day 10) surprisingly revealed a significantly lower form of the disease (FIG. 1). The mean maximal score of mice vaccinated with SDF-1α was 1.166±0.18 compared to 2.5±0.24, 2.66±0.23, and 2.33±0.36 in control groups vaccinated with either an empty vector, plasmid DNA encoding β-actin or PBS, respectively (FIG. 1, p<0.01). Moreover, whereas all mice from this group went into remission within 12-13 days of disease onset, all control mice continued to develop a semi-chronic form of EAE that persisted for more than 3 weeks.

Histological analysis confirmed the differences in the clinical manifestation of the disease. As shown in FIGS. 2A-D, mice subjected to plasmid DNA encoding SDF-1α displayed a lower histological score (FIG. 2D, 0.366±0.1, p<0.001) compared to control EAE mice treated with PBS (FIG. 2B, 2.2±0.4, p<0.001) or with β-actin encoding plasmid (FIG. 2C, 2.4±0.3, p<0.001). In addition, no SDF-1α-specific antibody titer could be recorded in the SDF-1α treated mice EAE suppressed mice (data not shown).

### Example 2

### SDF-1α Neutralizing Antibodies Reversed the Effect of DNA Vaccine Encoding SDF-1α

Materials and Experimental Procedures

Administration of Monoclonal Antibodies Neutralizing SDF-1α DNA Vaccines

Just after the onset of active EAE disease (day 10), C57BL\6 female mice were separated into five groups based on the severity of the disease (6 per group). On days 11, 13, 15, and 17 after the induction of disease, EAE mice were injected intramuscularly (i.m.) with DNA vaccine encoding SDF-1α. 2-5 hours later, the mice were also administered with anti-SDF-1α mAb (i.v) at a concentration of 50 μg/mouse (R&D Systems, Inc. Minneapolis, Minn.) or with a control antibody (isotype-matched control IgG, Sigma, St. Louis, Mo.). An observer blind to the experimental protocol then monitored the development and progression of the disease.

Results

As clearly shown in FIG. 3, repeated administration of SDF-1α mAb (but not isotype-matched control IgG) reversed the therapeutic effect of SDF-1α vaccine on EAE as scored on day 20. Control EAE mice had an EAE score of 2.33±0.36, empty plasmid had an EAE score of 2.83±0.18 and DNA vaccines encoding SDF-1α had an EAE score of 0.83±0.18. Treatment of EAE mice with DNA vaccines encoding SDF-1α along with repeated administration of SDF-1α mAb had an EAE score of 2.83±0.8, whereas subjection of these mice to control mAb resulted in an EAE score of 2.5±0.24. Taken together, these results suggest that DNA vaccines encoding SDF-1α function in a beneficial manner in the treatment of EAE.

### Example 3

### SDF-1α Possess a Dual Function in Regulation of EAE

Materials and Experimental Procedures

Administration of Monoclonal Antibodies Neutralizing SDF-1α after the Onset of EAE

Just after the onset of active EAE disease (day 10), C57BL\6 female mice were separated into three groups based on the severity of the disease (6 per group). On days 11, 13, 15, and 17 after the induction of disease, EAE mice were subjected to either PBS (i.m.), anti-SDF-1α mAb at a concentration of 50 μg/mouse (i.v., R&D) or control antibody (isotype-matched control IgG, Sigma, St. Louis, Mo.). An observer blind to the experimental protocol then monitored the development and progression of the disease. Results are shown as mean EAE score ±SE.

Administration of Monoclonal Antibodies Neutralizing SDF-1α Before the Onset of EAE

C57BL\6 female mice were separated into three groups based on the severity of the disease (6 per group). Starting on day 7 (after induction of EAE), 3-4 days prior to the onset of active EAE disease, these mice were subjected every other day to either PBS (i.m.), 50 μg/mouse anti-SDF-1α mAb (i.v., R&D) or control antibody (isotype-matched control IgG, Sigma, St. Louis, Mo.). An observer blind to the experimental protocol then monitored the development and progression of the disease. Results are shown as mean EAE score ±SE.

Results

As shown in FIG. 4A, mice subjected to anti-SDF-1α mAb just after the onset of EAE developed an exacerbated, long-term EAE (mean maximal score of 3±0.28) much more severe than mice subjected to control antibodies (mean maximal score of 2.166±0.18 for either control PBS or control antibody, p<0.03). These results suggest a possible role for SDF-1α as an anti-inflammatory chemokine in regulating ongoing EAE.

As evident from the results (FIG. 4B), administration of anti-SDF-1α mAb prior to the onset of the disease lead to a delay of 3-4 days in the start of active EAE. Yet, following its initiation, the severity of the disease reached a markedly higher score (day 18, 3.5±0.23) compared to the control groups (PBS or control antibody, 2.5±0.23 and 2.66±0.23, p<0.02, respectively).

It was previously reported that SDF-1α, produced by various residual cells within the central nervous system (CNS), is involved in directing the migration of leukocytes to these tissues [Bleul et al., J Exp Med (1996) 184:1101-9; Pelletier et al., Blood (2000) 96:2682-90]. These reports may partially explain why neutralization of SDF-1α before the onset of disease delays the initiation of the inflammatory process within the CNS. However, once active EAE has initiated, SDF-1α manifests a regulatory function. Taken together, these results suggest that the ongoing stage of EAE is a more relevant for novel therapeutic strategies involving SDF-1α.

### Example 4

### SDF-1α Functions as a Regulatory and Anti-Inflammatory Mediator

Materials and Experimental Procedures

Isolation and In-Vitro Activation of EAE Spleen Cells

Spleen cells were collected from mice 15 days post induction of EAE. Cells were cultured in a humidified 7.5% CO₂ atmosphere at 37° C. and stimulated with 50 μg/ml MOG₃₅₋₅₃ peptide. 10×10⁶ spleen cells were cultured in 24-well plates in the presence of Recombinant SDF-1α (rSDF-1α, R&D, Minneapolis, Minn.) or PBS for 72 hours. Supernatants were collected and analyzed by ELISA.

Isolation and In-Vitro Activation of Monocytes

Human monocytic (THP-1) cells were differentiated into macrophage-like cells by culturing 1×10⁶ THP-1 cells for 96 hours in 24-well plates in the presence of 30 nM PMA. Differentiation growth medium contained RMPI 1640 (Biological Industries, Kibbutz Beit-Haemek, Israel) supplemented with 5% FCS (Biological Industries, Kibbutz Beit-Haemek, Israel) and Penicillin Streptomycin (Biological Industries, Kibbutz Beit-Haemek, Israel). Cells were cultured in a humidified 7.5% CO₂ atmosphere at 37° C. At the end of differentiation, cell growth medium containing PMA was replaced by fresh RPMI medium supplemented with 7.5% FCS. 24 hours later, the adherent cells were washed and stimulated with 0.5 μg/ml LPS (Sigma, St. Louis, Mo.).

Peritoneal macrophages were isolated from naïve mice that had been injected intraperitoneally 5-7 days previously with 3 ml of thioglycolate broth (2.5%) (Sigma, St. Louis, Mo.). 1×10⁶ cells/well were plated in 24-well plates in a humidified 7.5% CO₂ atmosphere at 37° C. 24 hours later non-adherent cells were removed by washing the plates twice with PBS. The remaining adherent cells (macrophages) were stimulated with 0.5 mg/ml LPS (Sigma, St. Louis, Mo.).

Recombinant SDF-1α (rSDF-1α; R&D, Minneapolis, Minn.) was added at different concentrations (0-100 ng/ml) to LPS-activated peritoneal macrophages or THP-1 derived macrophages. 24 hours later supernatants were collected and analyzed by ELISA.

Isolation and In-Vitro Activation of T Helper Cells

CD4⁺ T cells from spleens of naïve C57BL/6 mice were isolated by incubation with CD4+ T cell biotin antibody and then purified with CD4+ T cell anti-biotin microbeads (Miltenyi Biotec). These cells were subjected to anti-CD3-induced activation prior to subjection to different concentrations of SDF-1α (0-150 ng/ml). 48 hours later supernatants were collected and analyzed by ELISA.

ELISA

Secreted levels of IL-10, IL-12, TNF-α, TGF-β and IL-2 were each measured by commercially available ELISA kits: IL-10 (BioLegend, San Diego, Calif.), IL-12 (Bender Medical Systems, Vienna, Austria), TNF-α (Bender Medical Systems, Vienna, Austria), TGF-β (R&D Systems, Minneapolis, Minn.) and IL-2 concentration (BioLegend, San Diego, Calif.).

Results

As illustrated in FIGS. 5A-B, supplementing EAE spleen cells (T cells, B cells and macrophages) with up to 100 ng SDF-1α induced an elevated dose-dependent production of IL-10 (FIG. 5A, p<0.01) along with a significant reduction in IL-12 and TNF-α production (FIGS. 5B-C, p<0.01).

Similar results were documented for monocytes. Thus, supplementing peritoneal monocytes with SDF-1α initiated a dose-dependent elevation in IL-10 production in conjunction with a reduction in TNF-α and IL-12 production (FIGS. 5D-F, p<0.01).

A significant, dose-dependent elevation in IL-10 production (FIG. 5G, p<0.01) was also observed in T cell cultures supplemented with 100 ng SDF-1α. Addition of SDF-1α to T cells was also accompanied by a significant reduction in TNF-α production (FIG. 5H, p<0.01) and a dose-dependent increase in IL-2 production (FIG. 5I). The increased production of IL-2 by T cells suggested that the increased production of IL-10 resulted, in part, by the differential proliferation of IL-10-producing cells, but it does not explain the reduced levels of TNF-α produced by the cultured cells. Thus, these results suggest a role for SDF-1α as a regulatory mediator.

Taken together, these results suggest that SDF-1α directs the functional polarization of macrophages and T cells into high-IL-10, low-inflammatory mediator-producing cells.

### Example 5

### CXCR4 Functions as a Receptor for SDF-1α

Materials and Experimental Procedures

Neutralization of CXCR4 by Specific mAb

THP-1 monocytic cells (cell line isolated from acute monocytic leukemia, ATCC Accession NO. TIB-202) were grown in cell medium containing: RPMI 1640 medium with 2 mM L-glutamine adjusted to contain 1.5 g/L sodium bicarbonate, 4.5 g/L glucose, 10 mM HEPES and 1.0 mM sodium pyruvate. Medium was supplemented with 0.05 mM 2-mercaptoethanol, 90%; and fetal bovine serum, 10%. CXCR4 was blocked by specific monoclonal antibodies (R&D) at a concentration of 20 μg/ml for 30 minutes.

Cell Migration Assay

10⁶ THP-1 cells were loaded into the upper chamber of a 6.5-mm diameter, 5-μm-pore polycarbonate Transwell culture insert (Costar, Cambridge, Mass.). CXCR4 mAb (R&D) was added to the THP-1 cells (in the upper chamber) at a concentration of 20 μg/ml for 30 minutes. The lower chamber contained 10 ng/ml rSDF-1 (R&D Systems, Minneapolis, Minn.) or 100 ng/ml SDF-1-Ig fusion protein. Cells were permitted to migrate for 2 hours at 37° C. in 7.5% CO₂. Cells that migrated were collected and counted using a FACSCalibur (BD Biosciences). The percentage of cell migration was calculated as the number of cells that migrated to the lower chamber divided by the number of cells originally plated in the upper chamber.

Flow Cytometry (FACS) Analysis

Flow cytometry analysis was conducted according to the protocol previously described by Schif-Zuck et al. [Schif-Zuck et al., J Immunol (2005) 174:4307-4315]. Briefly, 10⁶ cells were suspended in 1000 μl dyeing buffer containing an anti CD4-APC (BioLegend, San Diego, Calif.) labeled for 5 minutes on ice. The cells were washed three times in dyeing buffer and resuspended in 100 μl 1% PFA and transferred into FACS tubes.

Intracellular staining of IL-10 was conducted using PE labeled anti-mouse IL-10 (BD Biosciences).

Results

Blocking CXCR4, the primary receptor for SDF-1α, of THP-1 monocytic cells by specific mAb (R&D), under saturating conditions, inhibited about 50% of SDF-1α-induced IL-10 (FIG. 6A). CXCR4 mAb also inhibited about 80% of SDF-1α-induced migration of these cells (FIG. 6B). Together these results suggest the involvement of CXCR4 in SDF-1α-induced IL-10 production.

### Example 6

### SDF-1α-Ig Fusion Protein Maintained the Functional Properties of SDF-1α

Materials and Experimental Procedures

Construction of SDF-1α-Ig

The nucleic acid vector encoding the SDF-1α-Ig fusion protein of the present invention was constructed as follows: cDNA encoding the constant region (Hinge-CH2-CH3, SEQ ID NO: 3) of human IgG1 heavy chain was generated by RT-PCR of RNA extracted from LPS and IL-4 activated peripheral blood mononuclear cells (PBMC) using the primers: sense, 5′ ctcgagCCCAAATCTTGTGACAAAAC 3′ (SEQ ID NO: 7) and antisense: 5′ gggcccTTTACCCGGGGACAGGGAGA 3′ (SEQ ID NO: 8). The PCR product was digested with XhoI and ApaI and ligated into mammalian expression/secretion vector pSecTag2/Hygro B (Invitrogen Life Technologies, San Diego, Calif.). cDNA encoding mouse SDF-1α (GenBank Accession Nos. BC006040 or E09670, SEQ ID NO: 9) was generated by RT-PCR of RNA extracted from mouse splenocytes using the primers: sense, 5′ gctagcATGGACGCCAAGGTCGTCGC 3′ (SEQ ID NO: 11) and antisense, 5′ ctcgagCTTGTTTAAGGCTTTGTCC 3′ (SEQ ID NO: 12). The PCR product was digested with NheI and XhoI and following sequence verification, the amplified PCR product was subcloned into the pSec-Tag2 vector (Invitrogen, San Diego, Calif.) upstream of the human IgG1 fragment to create a fusion protein SDF-1α-Ig (SEQ ID NO: 13).

Since alterations in the amino acid sequence at the N-terminus of chemokines might change their properties, NheI was selected for the cloning procedure and the original murine kappa chain leader sequence found in pSecTag2/Hygro B was replaced by mouse SDF-1α leader sequence: The fused fragments were sequenced by dideoxynucleotide sequencing (Sequenase version 2; Upstate Biotechnology, Cleveland, Ohio).

Expression and Purification of SDF-1α-Ig Fusion Protein

The pSec-SDF-1α-IgG plasmid was co-transfected into DG44 Chinese hamster ovary (CHO) cells that have a double deletion for the dihydrofolate reductase (DHFR) gene (DG44 CHO DHFR^{-/-} cells, provided by Dr. Lawrence Chasin from Columbia university, USA, ATCC Accession No. CRL-9096), with CHO DHFR minigene vector, which transfects DHFR-deficient CHO cells with high efficiency, using jet PEI (Polypluse transfection—Illkirch Cedex, France) according the manufacturer's protocol. Stably transfected cells were selected in a culture medium (MEM-alpha) containing hygromycine (200 μg/ml) and increasing doses of methotrixate (2.5 nM to 0.1 mM). The fusion protein was expressed as a disulphide-linked homodimer similar to IgG1, and it had a molecular weight of approximately 72 kDa consisting of two identical 36 kDa subunits. The fusion protein was purified from the culture medium by High-Trap protein G affinity column (BD Biosciences, Piscataway, N.J.) and verified by western blot analysis using mouse anti-hIg (Jackson ImmunoResearch Laboratories, West Grove, Pa.) as primary antibody and donkey anti-mouse HRP-conjugated antibody (Jackson ImmunoResearch Laboratories, West Grove, Pa.) as secondary antibody.

β-actin-Ig was constructed and purified under the same conditions as described hereinabove for the purpose of a control peptide.

Cell Migration Assay

Cell migration assay was conducted as described hereinabove in Example 5, with the following modifications: The lower chamber of the Transwell culture insert (Costar, Cambridge, Mass.) was supplemented with culture media, 10 ng/ml rSDF-1α (R&D Systems, Minneapolis, Minn.), 100 ng/ml SDF-1α-Ig fusion protein or 100 ng/ml β-actin-Ig.

ELISA

Explained in detail in Example 4.

Results

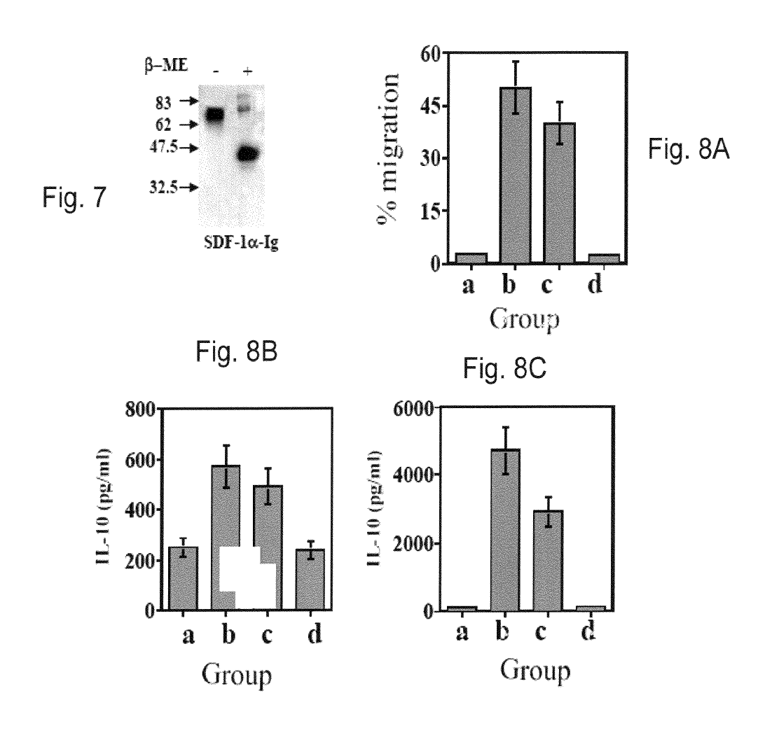

The chimeric peptide SDF-1α-IgG (Fc) was expressed (FIG. 7). In order to verify that the chimeric peptide maintained the functional properties of SDF-1α, its ability to attract human THP-1 monocytic cell line cells in a Transwell system (FIG. 8A, p<0.001) was verified, as well as in Jurkat cells (data not shown), and its ability to elicit IL-10 production in LPS-activated peritoneal macrophages (FIG. 8B) and in primary spleen cells (T cells) undergoing antigen-specific in vitro activation (FIG. 8C) was also verified. The fusion SDF-1α-IgG protein of the present invention, as well as the commercially available rSDF-1α (R&D, Minneapolis, Minn.), significantly (FIGS. 8B-C, p<0.01) induced IL-10 production in these cells as measured by ELISA.

### Example 7

### SDF-1α-Ig Fusion Protein Suppresses Ongoing EAE

Materials and Experimental Procedures

Administration of SDF-1α-Ig Fusion Protein

C57BL\6 female mice were subjected to active induction of EAE (MOGp₃₅₋₅₅/CFA). Just after the onset of active EAE disease (day 10), these mice were separated into three groups based on the severity of the disease (6 per group). On days 11, 13, 15 and 17 these mice were injected (i.v.) with 200 μg SDF-1α-Ig, control peptide β-actin-Ig or PBS.

### Histopathology

On day 20 (from induction of EAE), three representative mice from each group (as described above) were euthanized and lumbar spinal cords were removed. Histopathology analysis was performed as detailed in Example 1 hereinabove.

Immunohistochemistry

On day 20 (from induction of EAE), three representative mice from each group (as described above) were euthanized. Lumbar spinal cords were dissected, fixed in 4% paraformaldehyde, dehydrated and embedded in paraffin. 5 μm thick sections were mounted on Superfrost slides, deparaffinized, and blocked using normal Donkey serum (Jackson ImmunoResearch Laboratories, West Grove, Pa.). Slides were subjected to immunohistochemistry analysis using goat anti-IL-10 antibody (R&D Systems, Minneapolis, Minn.) as a primary antibody and donkey anti-goat biotinylated antibody (Jackson ImmunoResearch Laboratories, West Grove, Pa.) as a secondary antibody. Streptavidin-conjugated peroxidase (Zymed Laboratories Inc., San Francisco, USA). AEC (Zymed Labratories Inc., San Francisco, USA) was used as a substrate.

Results

As shown in FIG. 9, repeated administration of SDF-1α-Ig fusion protein, but not a control β-actin-Ig fusion protein, effectively and rapidly suppressed EAE. All control mice continued to develop a semi-chronic form of EAE that persisted for more than 4 weeks (on day 20, the mean maximal score of non treated EAE mice was 2.1±0.166 and β-actin-Ig treated EAE mice was 2.3±0.26, whereas all SDF-1α-Ig-treated EAE mice went into remission within 7-8 days (on day 20, the mean maximal score documented was 0.166±0.16, p<0.001).

Histological analysis conducted on lumbar spinal cord sections on day 20 verified the clinical results (FIGS. 10A-D and table). The mean histological score of SDF-1α-Ig-treated EAE mice was 0.4±0.3, whereas the mean histological scores of EAE induced mice or β-actin-Ig treated EAE mice were 2.3±0.3 and 2.1±0.3, respectively. Sequential sections were also subjected to immunohistochemical analysis of IL-10, showing the persistence of IL-10^{high} cells within the few perivascular infiltrates in sections from SDF-1α-Ig-treated mice, but not in control groups (FIGS. 10E-H).

### Example 8

### SDF-1α-Ig Fusion Protein Functions as an Anti-Inflammatory Mediator

Materials and Experimental Procedures

Administration of SDF-1α-Ig Fusion Protein

C57BL\6 female mice were subjected to active induction of EAE (MOGp₃₅₋₅₅/CFA). Just after the onset of active EAE disease (day 10), these mice were separated into three groups based on disease severity (6 per group). On days 11 and 13 these mice were injected (i.v.) with 200 μg SDF-1α-Ig, control peptide β-actin-Ig or PBS.

ELISA

On day 15 (from induction of EAE), three representative mice from each group (as detailed above) were euthanized and their spleens were removed. Splenocytes were cultured in the presence of their target antigen (MOGp₃₅₋₅₅). 24 hours later the levels of IL-10, IL-4, IL-12, TNF-α, TGF-β, IL-17 and IL-23 were measured by ELISA (described in more detail in Example 4 hereinabove).

Immunohistochemistry

On day 15 (from induction of EAE), three representative mice from each group (as explicated above) were euthanized. Spleens were dissected, fixed in 4% paraformaldehyde, dehydrated and embedded in paraffin. 5 μm thick sections were mounted on Superfrost slides, deparaffinized, and blocked using normal Donkey serum (Jackson ImmunoResearch Laboratories, West Grove, Pa.). Slides were subjected to immunohistochemistry analysis using goat anti-IL-10 antibody (R&D Systems, Minneapolis, Minn.) as a primary antibody and donkey anti-goat biotinylated antibody (Jackson ImmunoResearch Laboratories, West Grove, Pa.) as a secondary antibody. Streptavidin-conjugated peroxidase (Zymed Labratories Inc., San Francisco, USA). AEC (Zymed Labratories Inc., San Francisco, USA) was used as a substrate.

Intracellular FACS Analysis

On day 15 (from induction of EAE), three representative mice from each group (as detailed above) were euthanized. Spleens were dissected, cultured with the target antigen (MOGp₃₅₋₅₅), and after 3 days of incubation, were subjected to flow cytometry (FACS) analysis of intra-cellular staining for IL-10.

Intracellular staining of IL-10 was conducted using PE labeled anti-mouse IL-10 (BD Biosciences). CD4+ T cell staining was conducted using Flow Cytometry (FACS) analysis.

Results

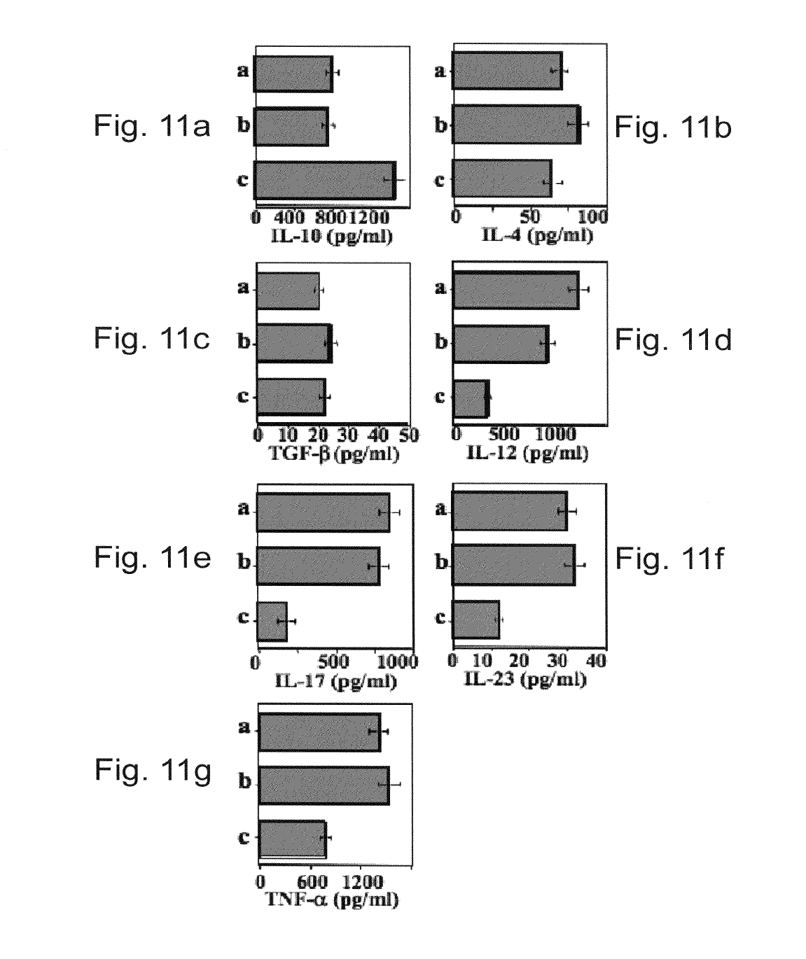

Spleen cell cultures derived from SDF-1α-Ig-treated EAE mice displayed a significantly higher level of IL-10 (FIG. 11A, 1450±170 pg/ml) compared to EAE induced mice or β-actin-Ig treated EAE mice (790±70 and 750±65, respectively, p<0.01). These results were accompanied by reduced production of macrophage proinflammatory mediators IL-12, IL-17, IL-23 and TNF-α. IL-12 production by SDF-1α-Ig-treated EAE mice was significantly lower (FIG. 11D, 330±27 pg/ml) compared to EAE induced mice or β-actin-Ig treated EAE mice (1230±140 and 920±80, respectively, p<0.01). Similarly, IL-23 production by SDF-1α-Ig-treated EAE mice was significantly lower (FIG. 11F, 13±1.2 pg/ml) compared to EAE induced mice or β-actin-Ig treated EAE mice (30±4.3 and 32±3.1, respectively, p<0.01), as well as TNF-α production by SDF-1α-Ig-treated EAE mice (FIG. 11G, 780±55 pg/ml) compared to EAE induced mice or β-actin-Ig treated EAE mice (1420±60 and 1540±130, respectively, p<0.01). A significant reduction in IL-17 production was recorded for SDF-1α-Ig-treated EAE mice (FIG. 11E, 160±22 pg/ml) compared to EAE induced mice or β-actin-Ig treated EAE mice (820±115 and 780±95, respectively, p<0.001). No apparent changes in TGF-β (FIG. 11C) or IL-4 (FIG. 11B) secretion levels were noted. Thus, therapy with SDF-1α-Ig promotes anti-inflammatory cytokine production, particularly IL-10, while blocking the production of proinflammatory cytokines, including those directing the polarization of Th1 and Th17 cells, particularly the Th17 cytokine IL-17.

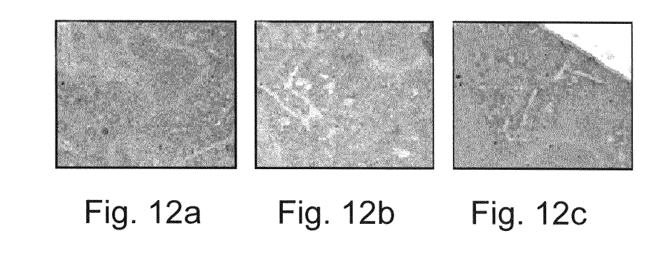

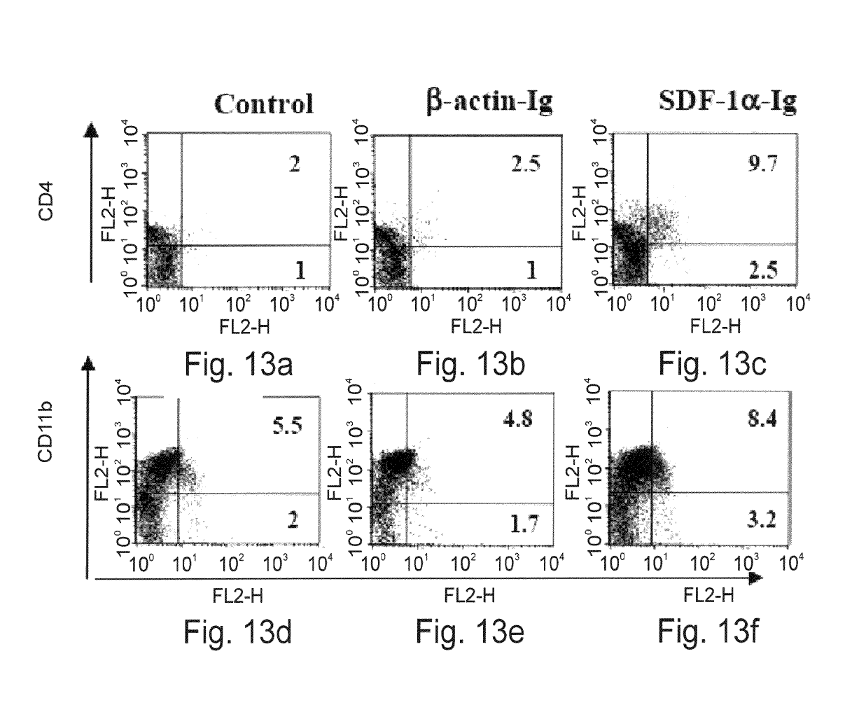

Immunohistochemical analysis of representative spleen sections also revealed high IL-10 expression in SDF-1α-Ig-treated mice (FIG. 12C) compared to control mice (FIG. 12A-B). Furthermore, intracellular FACS analysis, conducted on samples of cultured spleen cells from these mice, clearly showed a significant increase in IL-10 expression in CD4+ T cells from SDF-1α-Ig-treated mice (FIG. 13E, 9.7%) compared to EAE mice or β-actin-Ig-treated EAE mice (FIGS. 13A and C, 2% and 2.5%, respectively). Similar results were observed in IL-10 expression in macrophages (CD11b⁺) from SDF-1α-Ig-treated mice (FIG. 13F, 8.4%) compared to EAE mice or β-actin-Ig-treated EAE mice (FIGS. 13B and D, 5.5% and 4.8%, respectively).

### Example 9

### SDF-1α-Ig Fusion Protein has a Long Term Effect on Suppression of EAE

Materials and Experimental Procedures

Administration of SDF-1α-Ig Fusion Protein

C57BL\6 female mice were subjected to active induction of a long-term form of disease using the encephalitogenic peptide. Specifically, mice where immunized twice with MOGₚ₃₅₋₅₅/CFA (on days 0 and 7). Just after the onset of active EAE disease (day 10), these mice were separated into three groups based on the severity of the disease (6 per group). Twice a week these mice were injected (i.v.) with 200 μg SDF-1α-Ig, control peptide β-actin-Ig or PBS and were monitored for the development and progression of disease by an observer blind to the experimental protocol.

Proliferation Assay

Just before the peak of disease (day 24) lymph node cells (primary T cells) were isolated from the draining lymph nodes of primed mice (control, β-actin and SDF-1α-Ig treated mice) and cultured (5×10⁵ cells/well) in 96-well flat-bottomed plates (in triplicates) in the presence or absence of MOGp35-55. Cultures were incubated for 72 hours in a humidified 7.5% CO₂ atmosphere at 37° C. and [³H]-thymidine (1 μCi/well) was added for the last 16 hours of incubation. Cultures were harvested and counted. The proliferative response was expressed as stimulation index (SI): mean cpm of triplicates in the presence of antigen over mean cpm of triplicates in the absence of antigen (SD≦10%).

ELISA

Just before the peak of disease (day 24) primary T cells from the cervical lymph nodes of control, β-actin and SDF-1α-Ig treated mice were removed and subjected to MOGp35-55 induced activation. 24 hours later the level of IL-2 secretion was measured by ELISA (described in more detail in Example 4 hereinabove).

FACS Analysis

Just before the peak of disease (day 24) primary T cells from the cervical lymph nodes of control EAE mice, β-actin-Ig treated EAE mice and SDF-1α-Ig treated EAE mice were removed. The expression of Annexin V and PI in CD4+ T cells was analyzed by flow cytometry using FITC-rh Annexin V (a protein which exhibits antiphospholipase activity and binds to phosphatidylserine, Bender MedSystems, Vienna, Austria) and PI-propidium iodide (which allows the discrimination of apoptotic cells by binding to broken DNA pieces).

Results

As shown FIG. 14A, a marked long-lasting suppression of EAE is achieved by treatment with SDF-1α-Ig. On day 40, SDF-1α-Ig treated mice displayed a mean EAE score of 0.33±0.16 compared to 2.66±0.3 and 2.83±0.5 in β-actin-Ig or PBS treated mice, respectively (p<0.001).

Previously, it has been shown that SDF-1α is capable of inducing CD4+ T cell apoptosis via up-regulation of the Fas (CD95)/Fas ligand (CD95L) pathway [Colamussi et al., J Leukoc Biol (2001) 69: 263-70]. To elucidate whether administration of SDF-1α-Ig induces antigen specific CD4+ T cell apoptosis, primary T cells from the cervical lymph nodes of treated and control EAE mice were obtained just before the peak of disease (day 24) and were subjected to in vitro activation in the presence of their target determinant. As illustrated in FIGS. 14B-C, no significant difference in IL-2 production or proliferative response was recorded for T cells isolated from SDF-1α-Ig treated mice compared to mice treated with β-actin-Ig or PBS. Similarly, no major difference was recorded in the level of apoptotic cells, PI-Annexin V+ expressing CD4+ T cells (FIGS. 14D-F). Taken together, these results indicate that SDF-1α therapy suppresses EAE without inducing a significant alteration in T cell proliferation and apoptosis rate. It should be noted that under controlled in vitro conditions, supplementation of SDF-1α to anti-CD3 activated naïve T cells led to a significant increase in IL-2 production (see FIG. 5I).

### Example 10

### Antigen-Specific T Cells from Protected Donors Suppress EAE by Adoptive Transfer in an IL-10 Dependent Manner

Materials and Experimental Procedures

Administration of SDF-1α-Ig Fusion Protein to Donor Mice

C57BL\6 female mice were subjected to active induction of EAE (MOGp₃₅₋₅₅/CFA). Just after the onset of active EAE disease (day 10), these mice were separated into three groups based on the severity of the disease (6 per group). On days 11 and 13 these mice were injected (i.v.) with 200 μg SDF-1α-Ig, control peptide β-actin-Ig or PBS.

Selection of T Cells from Donor Mice

On day 15 (from induction of EAE), three representative mice from SDF-1α-Ig treated EAE mice or β-actin-Ig treated EAE mice groups were selected as detailed above) and euthanized. Spleens were dissected, cultured with the target antigen (MOGp₃₅₋₅₅), and after 3 days of incubation, CD4+ T cells (as determined by FACS) were subjected to FACS analysis of intra-cellular staining for IL-10 (as explained in detail in Example 8). CD4+ IL-10^{high} T cells were selected.

Transfer of T Cells to EAE Induced Mice

CD4+ IL-10^{high} T cells (20×10⁶ cells/mouse) were adoptively transferred to EAE mice (6 mice per group), at the onset of disease (on day 12), as follows: recipient group administered T cells isolated from protected mice (SDF-1α-Ig treated EAE mice), recipient group administered T cells isolated from β-actin-Ig treated EAE mice or a recipient group injected with PBS. All groups were monitored for the development and progression of disease by an observer blind to the experimental protocol.

FACS Analysis

Before being transferred to EAE mice, donor derived IL-10^{high} T cells from SDF-1α-Ig treated mice were tested for the expression of CD25 and FOXp3 by FACS analysis using anti-mouse CD25 (BioLegend, San Diego, Calif.) and anti-mouse FOXp3 (BioLegend, San Diego, Calif.)

Proliferation Assay

Before being transferred to EAE mice, donor derived IL-10^{high} T cells were tested for their ability to suppress the proliferative response of antigen specific primary T cells from control EAE mice, when added at a ratio of 1:10. Primary T cells from control EAE mice (105 per well), from protected mice (10⁵ per well or 10⁴ per well), or combinations thereof were examined for proliferation. Proliferation assay was performed as indicated in Example 9.

Anti-IL-10 mAb (50 μg/ml, R&D Systems Inc., Minneapolis, Minn.) were added to the wells.

Administration of SDF-1α-Ig Fusion Protein to IL-10^{-/-} Mice

C57BL/6 IL-10^{-/-} mice were subjected to active induction of EAE (MOGp35-55/CFA) and just after the onset of disease (day 11) they were injected (i.v) with SDS-1α-Ig (on day 11, 13, 15 and 17)

Results

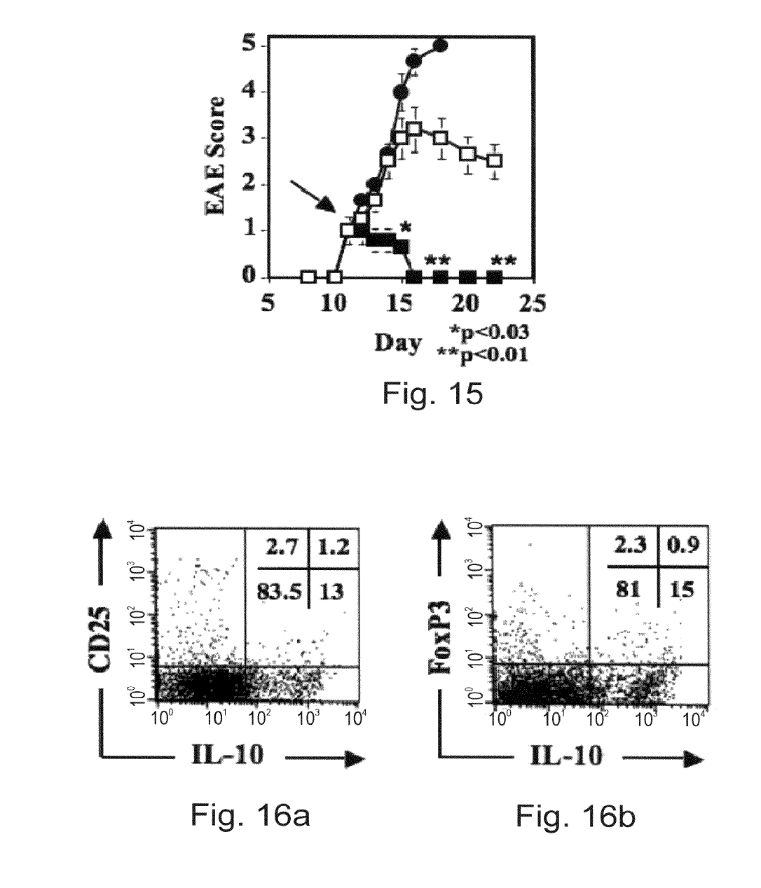

As evident from FIG. 15, transfer of antigen-specific primary T cells (as determined by FACS, FIG. 13E) from donor mice treated by SDF-1α-Ig to EAE mice elicited a rapid recovery of the disease (day 18 mean score 0±0, p<0.001). On the other hand, transfer of antigen-specific T cells from EAE donors treated by β-actin-Ig to EAE mice aggravated the severity of the disease (day 18 mean score 5±0) compared to control EAE mice treated by PBS (day 18 mean score 3±0.26, p<0.01).

Further analysis of T cells from protected donors (FIGS. 16A-B) revealed that a vast majority of these IL-10^{high} producing T cells are FOXp3⁻ (96%), CD25⁻ (86%) expressing cells. Thus, SDF-1α-Ig selects antigen specific regulatory CD4+ T cells that are IL-10^{high} CD25⁻FOXp3⁻ and are capable of suppressing EAE in adoptive transfer experiments.

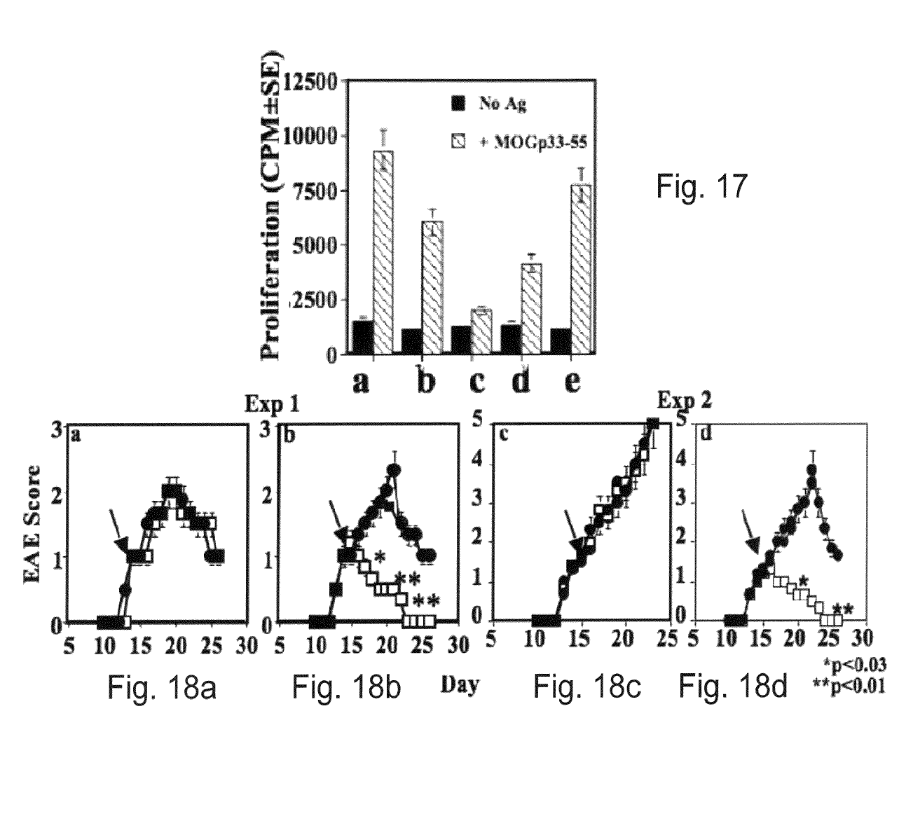

In an attempt to determine whether the regulatory function of these T cells is IL-10 dependent, experiments were performed to explore their ability to suppress the proliferative response of primary T cells from control EAE mice. As illustrated in FIG. 17, these regulatory T cells suppress the proliferative response of control primary cells responding to their MOGp35-55 target antigen (FIG. 17 lane d compared to lane a, 4100±340 CPM compared to 9320±860 CPM, respectively, p<0.001). This effect was reversed by anti-IL-10 mAb (lane e 7700±630 CPM, p<0.001).

Additionally, to determine whether the effect of SDF-1α-Ig based therapy is IL-10 dependent, the ability of SDF-1α-Ig to suppress EAE in IL-10^{-/-} mice (FIGS. 18A, 18C) compared to control IL-10^{+/+} EAE mice (FIGS. 18B, 18D) was carried out. As illustrated in FIG. 18B, while SDF-1α-Ig rapidly suppressed EAE in IL-10^{+/+} mice (day 22, mean EAE score for untreated mice was 2.5±0.66 compared to 0.5±0.13, for SDF-1α-Ig treated mice p<0.001), it had no effect on IL-10^{-/-} mice. Similar results were achieved in an equivalent experiment (FIGS. 18C-D).

### Example 11

### SDF-1α-Ig Redirects the Polarization of Antigen Specific Effector (Th1) Cells into IL-10 Producing Regulatory T Cells that Suppress EAE

Materials and Experimental Procedures

Selection and Stimulation of T Cells

Spleen cells (i.e. primary T cells) were collected from EAE donor mice 15 days post induction of EAE. Cells were cultured in a humidified 7.5% CO₂ atmosphere at 37° C. and stimulated with 50 μg/ml MOGp35-55 peptide. Primary T cells were subjected to two subsequent stimulation cycles in the presence of recombinant mouse IL-12 (R&D Systems Inc., Minneapolis, Minn.) and anti-IL-4 (R&D Systems Inc., Minneapolis, Minn.) neutralizing antibodies. T cells were then cultured in the presence or absence of SDF-1α-Ig (50 μg/ml).

Intracellular FACS Analysis

After 3 days of incubation, T cells were subjected to FACS analysis of intra-cellular staining for IL-4, IL-10, and IFN-γ.

Intracellular staining of IL-10 was accomplished using PE labeled anti-mouse IL-10 (BD Biosciences San Jose, Calif., USA). Intracellular staining of IL-4 was accomplished using anti-IL-10 PE labeled antibody (BD Biosciences San Jose, Calif., USA). Intracellular staining of IFN-γ was accomplished using anti-IFN-γ FITC labeled antibody (BD Biosciences San Jose, Calif., USA)

ELISA

After 3 days of incubation, T cell medium was collected and the levels of IL-4, IL-10, IFN-γ, TNF-α and TGF-β secretion were measured by ELISA (described in more detail in Example 4 hereinabove).

Transfer of T Cells to EAE Induced Mice

T cells which were subjected to two subsequent stimulation cycles in the presence of recombinant mouse IL-12 and anti-IL-4 neutralizing antibodies and which were then cultured in the presence or absence of SDF-1α-Ig (as indicated above), were adoptively transferred to EAE mice as follows: T cells (3×10⁶ cells/mouse) were adoptively transferred to EAE mice (6 mice per group), at the onset of disease (on day 10). All groups were monitored for the development and progression of disease by an observer blind to the experimental protocol.

Results

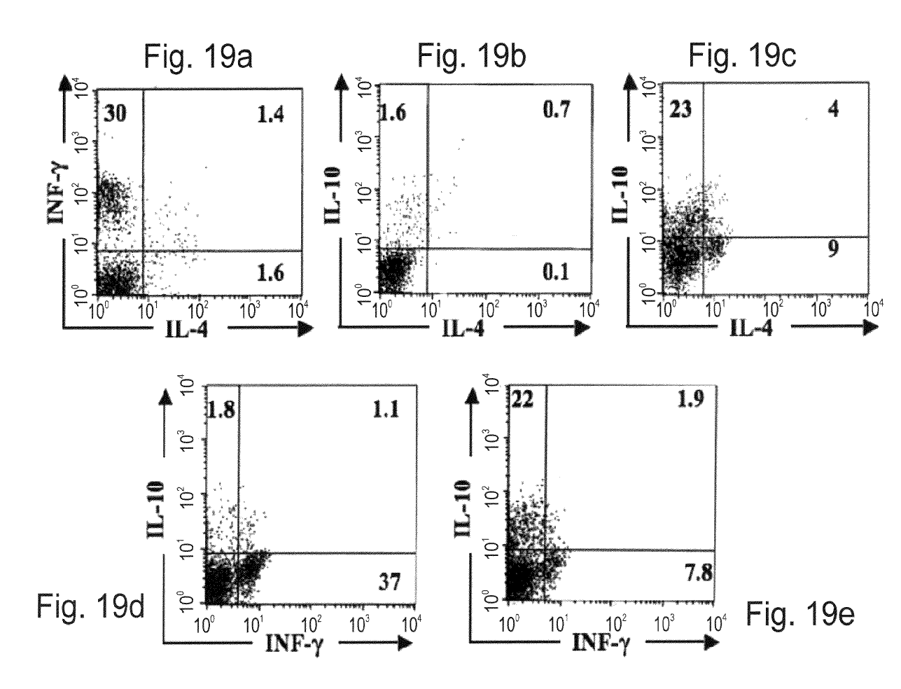

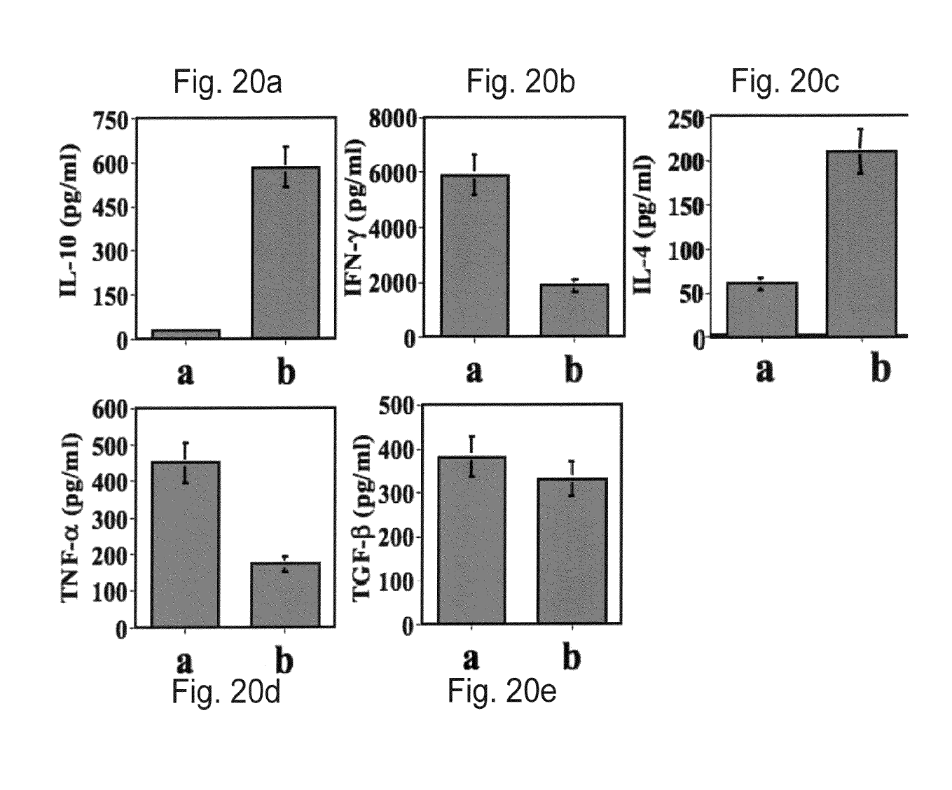

Intracellular FACS analysis showed that in the absence of SDF-1α-Ig, the vast majority of the polarized CD4+ T cells were IFN-γ^{high} IL-4^{low} Th1 cells (FIGS. 19A, 19B and 19D). However, addition of SDF-1α-Ig to the culture media (during the third stimulation cycle) redirected the polarization of a significant portion of these cells into IL-10^{high} IL-4^{low} cells (FIGS. 19B-C), resulting in a more than 10-fold increase in the level of IL-10 secretion (FIG. 20A, from 40±5 pg/ml in untreated cells to 580±25 pg/ml in SDF-1α-Ig treated cells, p<0.0001). Notably the relative number of IL-4^{high}IL-10^{low} CD4+ T cells also significantly increased (FIGS. 19B-C) resulting in the increase in IL-4 secretion (FIG. 20C, from 64±6 t pg/ml in untreated cells to 215±20 pg/ml in SDF-1α-Ig treated cells, p<0.001).

Further intracellular analysis of IL-10 versus IFN-γ in these cells clearly showed a highly significant increase in IL-10^{high}IFNγ^{low} CD4+ T cells (FIGS. 19D-E) accompanied by a decrease in IL-10^{low}IFNγ^{high} (FIGS. 19D-E) CD4+ T cells following re-selection in the presence of SDF-1α-Ig. These results were supported by a significant reduction in the secretion of IFN-γ (FIG. 20B, from 5850±430 pg/ml in untreated cells to 1930±210 pg/ml in SDF-1α-Ig treated cells, p<0.001). A significant decrease in TNF-α secretion was also recorded in SDF-1α-Ig treated cells (FIG. 20D, reduced from 440±55 pg/ml in untreated cells to 180±24 pg/ml in SDF-1α-Ig treated cells, p<0.001). No changes were observed in the levels of TGF-β secretion (FIG. 20F). Taken together, these results demonstrate an apparent shift from Th1 to IL-10 producing regulatory T cells in the presence of SDF-1α-Ig.

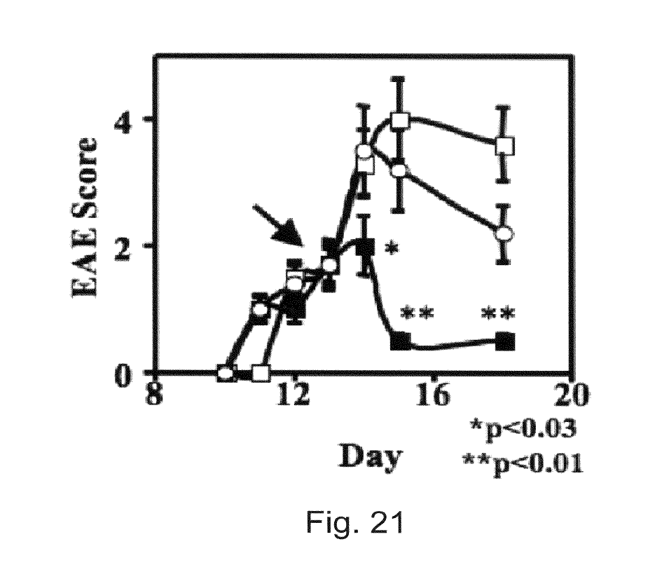

The therapeutic competence of these cells in adoptive transfer experiments was exemplified. As illustrated in FIG. 21, EAE mice that were treated, just after the onset of disease, with T cells that were selected in the presence of SDF-1α-Ig went into fast remission within 4-5 days (on day 15, mean maximal score was 0.66±0.3), whereas mice administrated PBS continued to develop a progressive form of disease (on day 15, mean maximal score was 3.3±0.6, p<0.01). Administration of effector T cells (from the same line) that were not co-cultured with SDF-1α-Ig to aggravated the severity of the disease (on day 15, mean maximal score was 4±0.3, p<0.05).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of treating Multiple Sclerosis in a subject in need thereof, the method comprising administering to the subject a therapeutically effective amount of SDF-1α, thereby treating Multiple Sclerosis in the subject.

2. A method of treating Multiple Sclerosis in a subject in need thereof, the method comprising:
(a) isolating T cells from the subject;
(b) subjecting said T cells to treatment with SDF-1α; and
(c) implanting said SDF-1α treated T cells into the subject, thereby treating Multiple Sclerosis in the subject.

3. The method of claim 2, wherein said subjecting is effected so as to upregulate secretion of IL-10 from said T cells.

4. The method of claim 2, wherein said T cells comprise regulatory T cells.

5. The method of claim 4, wherein said regulatory T cells comprise CD4⁺CD25⁻TOXp3⁻ T cells.

6. The method of claim 2, wherein said subjecting said T cells is further effected in a presence of IL-12 neutralizing antibody.

7. The method of claim 2, wherein said subjecting said T cells is further effected in a presence of anti-IL-4 neutralizing antibody.

8. (canceled)

9. An article of manufacture comprising SDF-1α and an anti-Multiple Sclerosis agent being packaged in a packaging material and identified in print, in or on said packaging material for use in the treatment of Multiple Sclerosis.

10. The method of claim 1, wherein said SDF-1α is capable of upregulating secretion of IL-10 from macrophages and T cells.

11. The method of claim 1, wherein the subject is undergoing an acute attack of Multiple Sclerosis.

12. The method of claim 1, wherein an amino acid sequence of said SDF-1α is attached to a heterologous amino acid sequence.

13. The method of claim 1, wherein the method does not comprise administering IL-2 or IL-4.

14. (canceled)

15. The article of manufacture of claim 9, wherein said anti-Multiple Sclerosis agent is not IL-2 or IL-4.

16. The method of claim 1, further comprising administering to the subject an additional anti-Multiple Sclerosis agent.

17. The article of manufacture or method of claim 9, wherein said anti-Multiple Sclerosis agent is selected from the group consisting of Interferon Beta 1a, Interferon Beta 1b, Glatiramer Acetate, Mitoxantrone, MethylPrednisolone, Prednisone, Prednisolone, Dexamethasone, Adreno-corticotrophic Hormone (ACTH) and Corticotropin.


SEQUENCE LISTING (15 sequences)

SEQ ID NO: 1 - DNA, 270 bp, Homo sapiens
  misc_feature: Human SDF-1 alpha coding sequence
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaa

SEQ ID NO: 2 - protein, 89 aa, Homo sapiens
  misc_feature: Human SDF-1 alpha
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 3 - DNA, 449 bp, Artificial sequence
  A polynucleotide encoding a portion of human IgG
ccgctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggt

SEQ ID NO: 4 - protein, 150 aa, Artificial sequence
  A portion of human IgG
PLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQV

SEQ ID NO: 5 - DNA, 716 bp, Artificial sequence
  A polynucleotide encoding human SDF-1 alpha fused to a portion of IgG
atgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagccgctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggt

SEQ ID NO: 6 - protein, 239 aa, Artificial sequence
  Human SDF-1 alpha fused to a portion of IgG
MNAKVVVVLVLVLTALCLSDGKPVSLSYRCPCRFFESHVARANVKHLKILNTPNCALQIVARLKNNNRQVCIDPKLKWIQEYLEKALNKPLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQV

SEQ ID NO: 7 - DNA, 26 bp, Artificial sequence
  Single strand DNA oligonucleotide
ctcgagcccaaatcttgtgacaaaac

SEQ ID NO: 8 - DNA, 26 bp, Artificial sequence
  Single strand DNA oligonucleotide
gggccctttacccggggacagggaga

SEQ ID NO: 9 - DNA, 270 bp, Mus musculus
  misc_feature: Murine SDF-1 alpha coding sequence
atggacgccaaggtcgtcgccgtgctggccctggtgctggccgcgctctgcatcagtgacggtaaaccagtcagcctgagctaccgatgcccctgccggttcttcgagagccacatcgccagagccaacgtcaagcatctgaaaatcctcaacactccaaactgtgcccttcagattgttgcacggctgaagaacaacaacagacaagtgtgcattgacccgaaattaaagtggatccaagagtacctggagaaagctttaaacaagtaa

SEQ ID NO: 10 - protein, 89 aa, Mus musculus
  misc_feature: Murine SDF-1 alpha
MDAKVVAVLALVLAALCISDGKPVSLSYRCPCRFFESHIARANVKHLKILNTPNCALQIVARLKNNNRQVCIDPKLKWIQEYLEKALNK

SEQ ID NO: 11 - DNA, 26 bp, Artificial sequence
  Single strand DNA oligonucleotide
gctagcatggacgccaaggtcgtcgc

SEQ ID NO: 12 - DNA, 25 bp, Artificial sequence
  Single strand DNA oligonucleotide
ctcgagcttgtttaaggctttgtcc

SEQ ID NO: 13 - DNA, 716 bp, Artificial sequence
  A polynucleotide encoding murine SDF-1 alpha fused to a portion of human IgG
atggacgccaaggtcgtcgccgtgctggccctggtgctggccgcgctctgcatcagtgacggtaaaccagtcagcctgagctaccgatgcccctgccggttcttcgagagccacatcgccagagccaacgtcaagcatctgaaaatcctcaacactccaaactgtgcccttcagattgttgcacggctgaagaacaacaacagacaagtgtgcattgacccgaaattaaagtggatccaagagtacctggagaaagctttaaacaagccgctcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggt

SEQ ID NO: 14 - protein, 239 aa, Artificial sequence
  Murine SDF-1 alpha fused to a portion of human IgG
MDAKVVAVLALVLAALCISDGKPVSLSYRCPCRFFESHIARANVKHLKILNTPNCALQIVARLKNNNRQVCIDPKLKWIQEYLEKALNKPLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQV

SEQ ID NO: 15 - protein, 21 aa, Artificial sequence
  Myelin oligodendrocyte glycoprotein (MOG 35-55) peptide
MEVGWYRSPFSRVVHLYRNGK